(19) United States
(12) Patent Application Publication
(10) Pub. No.: US 2018/0064335 A1
(43) Pub. Date: Mar. 8, 2018

(54) RETINAL IMAGER DEVICE AND SYSTEM WITH EDGE PROCESSING

(71) Applicant: Elwha LLC, Bellevue, WA (US)

(72) Inventors: Phillip Rutschman, Seattle, WA (US); Ehren Brav, Bainbridge Island, WA (US); Russell Hannigan, Sammamish, WA (US); Roderick A. Hyde, Redmond, WA (US); Muriel Y. Ishikawa, Livermore, CA (US); 3ric Johanson, Seattle, WA (US); Jordin T. Kare, San Jose, CA (US); Tony S. Pan, Bellevue, WA (US); Clarence T. Tegreene, Mercer Island, WA (US); Charles Whitmer, North Bend, WA (US); Lowell L. Wood, JR., Bellevue, WA (US); Victoria Y.H. Wood, Livermore, CA (US); Travis P. Dorschel, Issaquah, WA (US)

(73) Assignee: Elwha LLC, Bellevue, WA (US)

(21) Appl. No.: 15/698,147

(22) Filed: Sep. 7, 2017

Related U.S. Application Data
(63) Continuation-in-part of application No. 15/697,893, filed on Sep. 7, 2017.
Application No. 15/697,893 is a continuation-in-part of application No. 14/838,114, filed on Aug. 27, 2015.
Application No. 14/838,114 is a continuation-in-part of application No. 14/838,128, filed on Aug. 27, 2015.
Application No. 14/838,128 is a continuation-in-part of application No. 14/791,160, filed on Jul. 2, 2015, now Pat. No. 9,866,765.
Application No. 14/791,160 is a continuation-in-part of application No. 14/791,127, filed on Jul. 2, 2015.
Application No. 14/791,127 is a continuation-in-part of application No. 14/714,239, filed on May 15, 2015.
Application No. 14/714,239 is a continuation-in-part of application No. 14/951,348, filed on Nov. 24, 2015, now Pat. No. 9,866,881.
Application No. 14/951,348 is a continuation-in-part of application No. 14/945,342, filed on Nov. 18, 2015.
Application No. 14/945,342 is a continuation-in-part of application No. 14/941,181, filed on Nov. 13, 2015, now abandoned.
(60) Provisional application No. 62/180,040, filed on Jun. 15, 2015.
(60) Provisional application No. 62/156,162, filed on May 1, 2015.
(60) Provisional application No. 62/082,002, filed on Nov. 19, 2014.
(60) Provisional application No. 62/082,001, filed on Nov. 19, 2014.
(60) Provisional application No. 62/081,560, filed on Nov. 18, 2014.
(60) Provisional application No. 62/081,559, filed on Nov. 18, 2014.
(60) Provisional application No. 62/522,493, filed on Jun. 20, 2017.
(60) Provisional application No. 62/532,247, filed on Jul. 13, 2017.
(60) Provisional application No. 62/384,685, filed on Sep. 7, 2016.
(60) Provisional application No. 62/429,302, filed on Dec. 2, 2016.
(60) Provisional application No. 62/537,425, filed on Jul. 26, 2017.

(51) Int. Cl. A61B 3/12; A61B 3/14; A61B 3/00; G02B 27/10

(52) U.S. Cl. CPC A61B 3/12; A61B 3/145; G06T 7/12; G02B 27/10; A61B 3/0008

(57) ABSTRACT

In one embodiment, a machine-vision enabled fundoscope for retinal analysis includes, but is not limited to, an optical lens arrangement; an image sensor positioned with the optical lens arrangement and configured to convert detected light to retinal image data; computer readable memory; at least one communication interface; and an image processor communicably linked to the image sensor, the computer readable memory, and the at least one communication interface, the image processor programmed to execute operations including at least: obtain the retinal image data from the image sensor; generate output data based on analysis of the retinal image data, the output data requiring less bandwidth for transmission than the retinal image data; and transmit the output data via the at least one communication interface.

### PRIORITY CLAIM

This application claims priority to and/or the benefit of the following patent applications under 35 U.S.C. 119 or 120: U.S. Non-Provisional application Ser. No. 15/697,893 filed Sep. 7, 2017 (Docket No. 1114-003-014-000000); U.S. Non-Provisional application Ser. No. 14/838,114 filed Aug. 27, 2015 (Docket No. 1114-003-003-000000); U.S. Non-Provisional application Ser. No. 14/838,128 filed Aug. 27, 2015 (Docket No. 1114-003-007-000000); U.S. Non-Provisional application Ser. No. 14/791,160 filed Jul. 2, 2015 (Docket No. 1114-003-006-000000); U.S. Non-Provisional application Ser. No. 14/791,127 filed Jul. 2, 2015 (Docket No. 1114-003-002-000000); U.S. Non-Provisional application Ser. No. 14/714,239 filed May 15, 2015 (Docket No. 1114-003-001-000000); U.S. Non-Provisional application Ser. No. 14/951,348 filed Nov. 24, 2015 (Docket No. 1114-003-008-000000); U.S. Non-Provisional application Ser. No. 14/945,342 filed Nov. 18, 2015 (Docket No. 1114-003-004-000000); U.S. Non-Provisional application Ser. No. 14/941,181 filed Nov. 13, 2015 (Docket No. 1114-003-009-000000); U.S. Provisional Application 62/180,040 filed Jun. 15, 2015 (Docket No. 1114-003-001-PR0006); U.S. Provisional Application 62/156,162 filed May 1, 2015 (Docket No. 1114-003-005-PR0001); U.S. Provisional Application 62/082,002 filed Nov. 19, 2014 (Docket No. 1114-003-004-PR0001); U.S. Provisional Application 62/082,001 filed Nov. 19, 2014 (Docket No. 1114-003-003-PR0001); U.S. Provisional Application 62/081,560 filed Nov. 18, 2014 (Docket No. 1114-003-002-PR0001); U.S. Provisional Application 62/081,559 filed Nov. 18, 2014 (Docket No. 1114-003-001-PR0001); U.S. Provisional Application 62/522,493 filed Jun. 20, 2017 (Docket No. 1114-003-011-PR0001); U.S. Provisional Application 62/532,247 filed Jul. 13, 2017 (Docket No. 1114-003-012-PR0001); U.S. Provisional Application 62/384,685 filed Sep. 7, 2016 (Docket No. 1114-003-010-PR0001); U.S. Provisional Application 62/429,302 filed Dec. 2, 2016 (Docket No. 1114-003-010-PR0002); and U.S. Provisional Application 62/537,425 filed Jul. 26, 2017 (Docket No. 1114-003-013-PR0001). The foregoing applications are incorporated by reference in their entirety as if fully set forth herein.

### FIELD OF THE INVENTION

Certain embodiments of the invention relate generally to a retinal imager device and system with edge processing.

### SUMMARY

In one embodiment, a machine-vision enabled fundoscope for retinal analysis includes, but is not limited to, an optical lens arrangement; an image sensor positioned with the optical lens arrangement and configured to convert detected light to retinal image data; computer readable memory; at least one communication interface; and an image processor communicably linked to the image sensor, the computer readable memory, and the at least one communication interface, the image processor programmed to execute operations including at least: obtain the retinal image data from the image sensor; generate output data based on analysis of the retinal image data, the output data requiring less bandwidth for transmission than the retinal image data, and transmit the output data via the at least one communication interface.

In another embodiment, a process executed by a computer processor component of a fundoscope that includes an optical lens arrangement, an image sensor configured to convert detected light to retinal image data, and at least one communication interface, includes, but is not limited to, obtain the retinal image data from the image sensor; generate output data based on analysis of the retinal image data, the output data requiring less bandwidth for transmission than the retinal image data; and transmit the output data via the at least one communication interface.

In a further embodiment, a fundoscope includes, but is not limited to, means for obtaining retinal image data from an image sensor; means for generating output data based on analysis of the retinal image data, the output data requiring less bandwidth for transmission than the retinal image data; and means for transmitting the output data via the at least one communication interface.

### BRIEF DESCRIPTION OF THE DRAWINGS

Embodiments of the present invention are described in detail below with reference to the following drawings:

FIG. 1 is a perspective view of a retinal imager device with edge processing, in accordance with an embodiment;

FIG. 2 is a side view of an arrangement usable within a retinal imager device with edge processing, in accordance with an embodiment;

FIG. 3A is a zoom side view of anatomical structures of an eye positioned with a retinal imager device with edge processing, in accordance with an embodiment;

FIG. 3B is an illustration of non-uniform illumination of the retina, in accordance with an embodiment;

FIG. 4 is a component diagram of a retinal imager device with edge processing, in accordance with an embodiment; and

FIGS. 5-33 are block diagrams of processes implemented using a retinal imager device with edge processing, in accordance with various embodiments.

### DETAILED DESCRIPTION

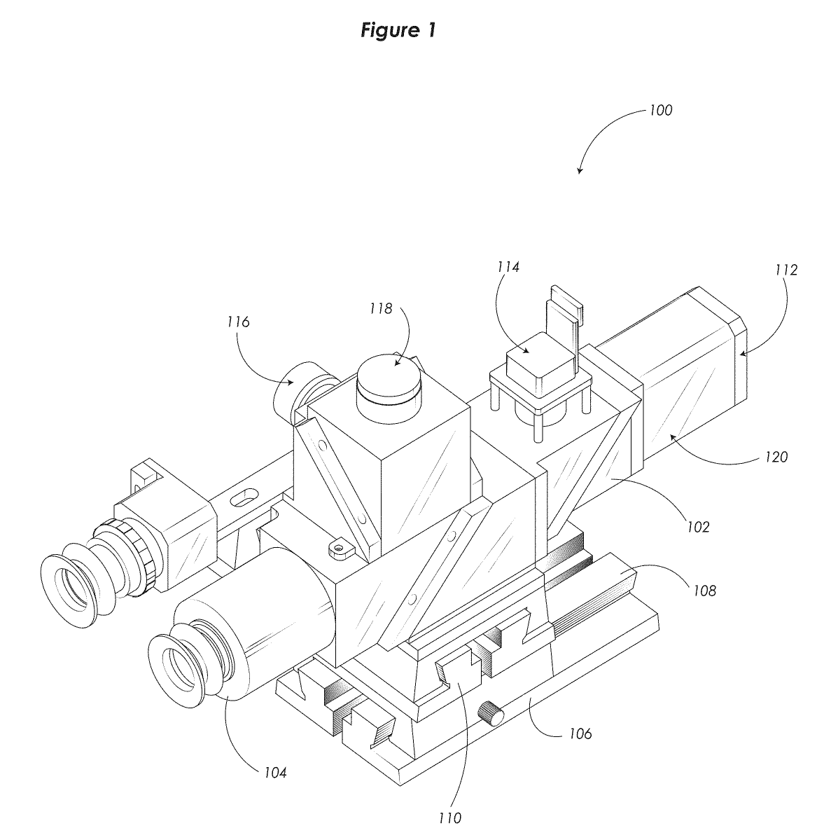

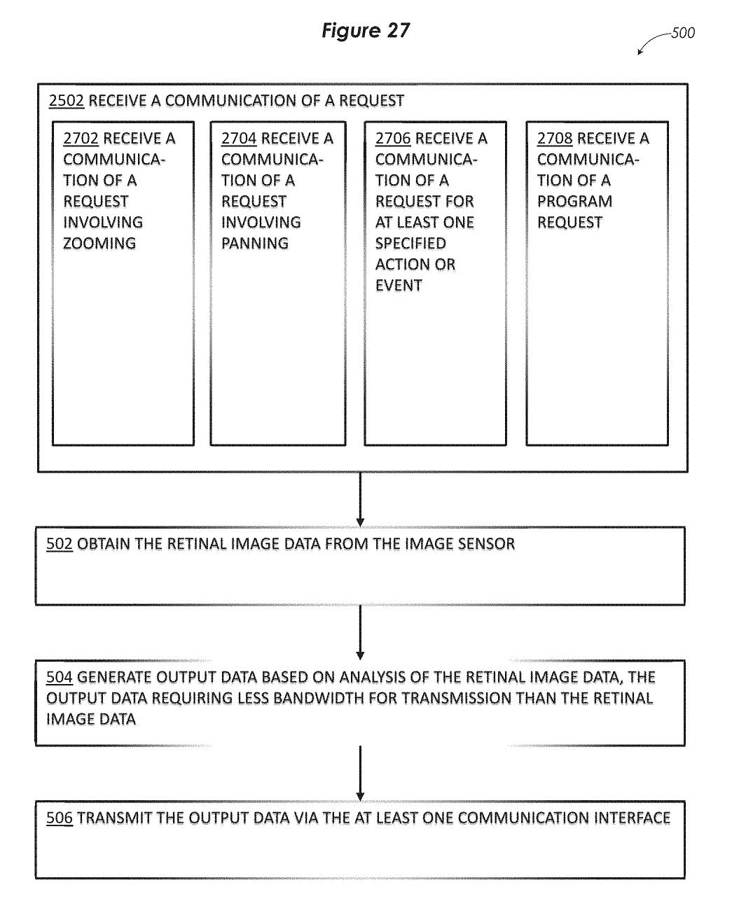

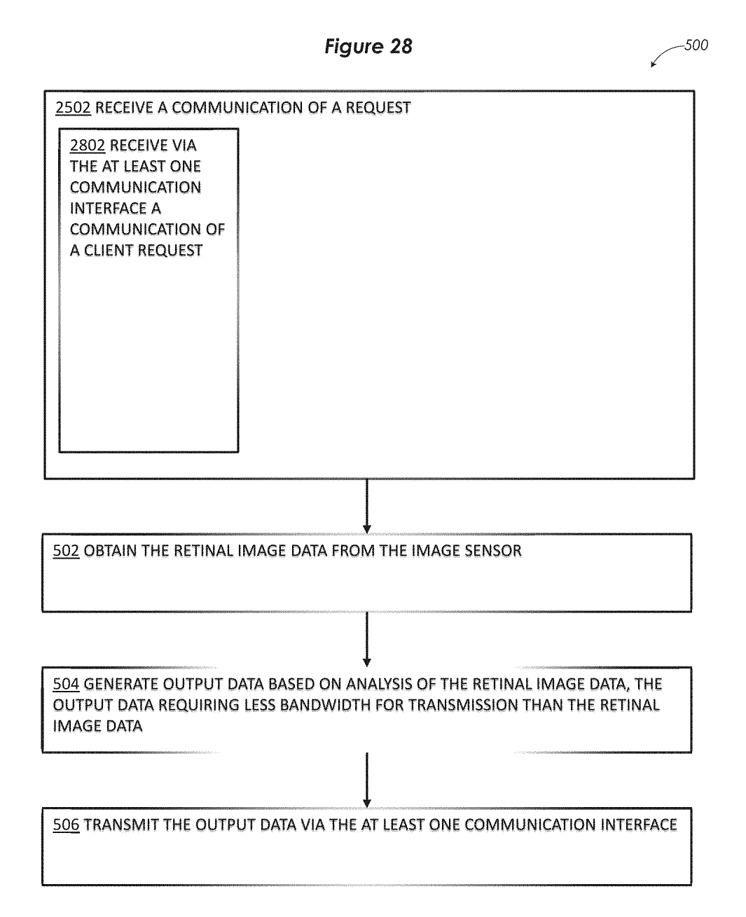

Embodiments disclosed herein relate generally to an imaging device and system with edge processing. Specific details of certain embodiments are set forth in the following description and in FIGS. 1-33 to provide a thorough understanding of such embodiments.

FIG. 1 is a perspective view of a retinal imager device 100 or fundoscope with edge processing, in accordance with an embodiment. The retinal imager device 100 provides machine vision for healthcare that enables minimally-obtrusive retinal monitoring and with extremely high visual acuity. For example, the retinal imager device 100 can perform rapid imaging of the retina with or without doctor or nurse supervision as and when needed and without requiring pupil dilation. Use contexts can include home, public, remote, health clinic, hospital, care facilities, outer space/space flights, or the like. For instance, the retinal imager device 100 can be usable/deployable on the International Space Station, Orion, or other crew spacecraft.

One particular embodiment includes a standalone compact self-contained device 100 including a housing 102, eye pieces 104, a mount bracket 106, a visible light emitting diode 118 (e.g., red, white, etc.), and/or an infrared light emitting diode 116 and an infrared imager 112 for enabling manual or automated retinal focus. Included within the retinal imager device 100, and which are discussed and illustrated further herein and which are partially or entirely concealed in FIG. 1, are an optical lens arrangement 120; an image sensor 114 positioned with the optical lens arrangement and configured to convert detected light to retinal image data; computer readable memory; at least one communication interface; and an image processor communicably linked to the image sensor 114, the computer readable memory, and the at least one communication interface, the image processor programmed to execute operations including at least: obtain the retinal image data from the image sensor 114; generate output data based on analysis of the retinal image data, the output data requiring less bandwidth for transmission than the retinal image data; and transmit the output data via the at least one communication interface.

The mount bracket 106 can be coupled or removably coupled to a support structure, such as a desk, table, wall, or other platform. The mount bracket 106 includes a z-axis track 108 and a y-axis track 110. The z-axis track 108 enables the housing 102 and the eyepieces 104 to move relative to a support structure along a z-axis (e.g., forward and aft). The y-axis track 110 enables the housing 102 to move relative to a support structure and relative to the eyepieces 104 along a y-axis (e.g., left and right). Thus, the housing 102 can move left and right between the eyepieces 104 to sample left and/or right eyes of a user. The housing 102 can further move forward and aft for user comfort or other adjustment.

In certain embodiments, the retinal imager device 100 includes one or more of the following properties or characteristics: approximately 10 mm eye-relief, polarizing optics to reduce stray light, operates over 450 nm to 650 nm, less than approximately seven microns spot size at the imager, annular illumination to mitigate stray light, adjustable focus for better than −4D to 4D accommodation, and/or an infrared channel with an approximately 850 nm light source and infrared imager for imaging approximately 10 mm of an eye for boresight alignment of the visible channel.

The retinal imager device 100 can assume a variety of forms and shapes and is not limited to the form illustrated in FIG. 1. For instance, the retinal imager device 100 can be incorporated into a wall, table, desk, kiosk, computer, smartphone, laptop, virtual reality headset, augmented reality headset, handheld device, pole mounted device, or other structure that may integrate, include, expose, or conceal part, most, or all of the structure depicted in FIG. 1. For example, the housing 102, the eyepieces 104, and the mount bracket 106 may be integrated into a personal health kiosk that conceals all but the eyepieces 104 to enable positioning of left and right eyes of a user with respect to the retinal imager device 100. Additionally, the retinal imager device 100 may omit the mount bracket 106 in favor of a non-movable mount bracket, a mount bracket that moves and pivots in additional directions (e.g., 360-degree rotation, tilt, y-axis movement, etc), or in favor of integration with a structure (e.g., a special purpose table that includes the retinal imager device 100 integrated thereon). Alternatively, the housing 102 can include two housings with redundant components for each of the left and right portions of the eyepieces 104. Moreover, the eyepieces 104 can include a single eye piece that is shared for left and right eyes of a user.

In one particular embodiment, the retinal imager device 100 is incorporated into or distributed between an eyebox, a laptop, monitor, phone, tablet, or computer that includes an interrogation signal device (e.g., tunable laser or infrared emitting device) and that includes a camera, which may be used to capture retinal imagery and/or detect eye position, rotation, pupil diameter, or vergence. The camera can comprise a co-aligned illumination device (e.g., red or infrared laser) and a plurality of high resolution cameras (e.g., 2-3). The display of the laptop or other device can auto-dim during imaging and output a visual indication or spot of focus for looking or staring at while the camera captures imagery of the retina or retinas of a user. An image processor coupled to the camera or cameras enables real-time on-board video acquisition, cropping, resizing, stitching, or other disclosed processing of imagery.

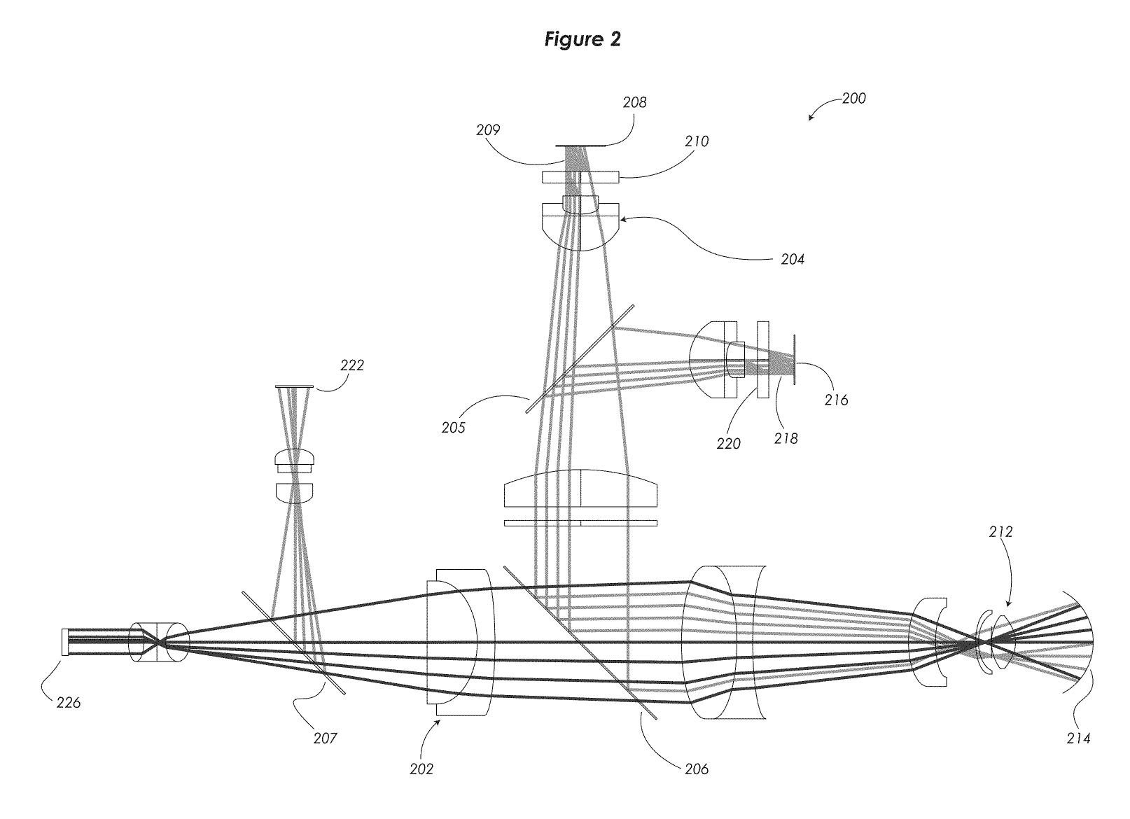

FIG. 2 is a side view of an arrangement 200 usable within a retinal imager device 100 or fundoscope with edge processing, in accordance with an embodiment. The arrangement 200 includes an imaging lens arrangement 202 aligned in a first axis, an illumination lens arrangement 204 aligned in a second axis that is perpendicular to the first axis, at least one polarizing splitter/combiner 206, an illumination LED 208 configured to emit light 209 for imaging, an image sensor 222 configured to convert detected light directed from a splitter 207 to retinal image data, and one or more masks 210 configured to obscure at least some of the light 209 of the illumination LED 208 prior to passing through the illumination lens arrangement 204, wherein the at least one polarizing splitter/combiner 206 is configured to redirect the light passing through the illumination lens arrangement 204 aligned in the second axis into the imaging optical lens arrangement 202 aligned in the first axis to illuminate at least one portion of the retina 214. In one particular embodiment, the imaging lens arrangement 202 is approximately 267 mm in length and an eye of an individual is positionable approximately 13 mm from an end of the imaging lens arrangement 202. In some embodiments, the arrangement 200 further includes an infrared LED 216 configured to emit infrared light 218 for positioning and/or focus determinations, a combiner 205, an infrared image sensor 226, and one or more infrared masks 220 configured to obscure at least some of the infrared light 218 of the infrared LED 216 prior to passing through the illumination lens arrangement 204, wherein the at least one polarizing splitter/combiner 206 is configured to redirect the infrared light 218 passing through the illumination lens arrangement 204 aligned in the second axis into the imaging optical lens arrangement 202 aligned in the first axis to illuminate at least one portion of the retina 214. In certain embodiments, the arrangement 200 further includes a microdisplay or is couplable to a computer, smartphone, laptop, or other personal device.

In one particular embodiment, the arrangement 200 operates as follows: the infrared LED 216 emits infrared light 218, which passes through one or more infrared masks 220, whereby at least some of the infrared light 218 is controllably blocked from further transmission. The infrared light 218 that passes by the one or more infrared masks 220 is directed into the illumination lens arrangement 204 via the combiner 205. The infrared light 218 then is directed into the imaging lens arrangement 202 via the polarizing splitter/combiner 206. The infrared light 218 then passes through the scattering elements of the eye 212 (e.g., of a person) before being reflected by the retina 214. The reflected infrared light 218 then returns through the imaging lens arrangement and is detected by the infrared imager 226. The infrared light 218 detected by the infrared imager 226 is used to determine whether the retina is centered and/or focused. The illumination LED 208 then emits light 209 for imaging that passes through the one or more masks 210 that block at least some of the light 209. The light 209 that passes through the one or more masks 210 then passes through the illumination lens arrangement 204 where it is directed into the imaging lens arrangement 202 via the polarizing splitter/combiner 206. The light 209 then passes through the scattering elements of the eye 212 before being reflected by the retina 214. The reflected light 209 then passes back through the imaging lens arrangement 202 and is directed by the splitter 207 to the image sensor 222. Retinal image data captured by the image sensor 222 can be stored, validated, and/or processed as disclosed herein. This process can be repeated as needed or requested, such as for both eyes of a person or for multiple individuals.

The arrangement 200 can be modified or substituted in whole or in part with one or more different arrangements to capture high resolution retinal imagery. For instance, any of the lenses, combination of lenses, position of lenses, shape of lenses, or the like may be modified as desired for a particular application. Also, the arrangement may include at least one additional imaging lens arrangement, and at least one additional image sensor positioned with the at least one additional imaging lens arrangement and configured to convert detected light to additional retinal image data. In this embodiment, the imaging lens arrangement 202 and the at least one additional imaging lens arrangement can have at least partially overlapping fields of view for capturing segments of a particular retina. Alternatively, the imaging lens arrangement 202 and the at least one additional imaging lens arrangement may have substantially parallel fields of view for capturing segments of a particular retina or for simultaneous capture of image data associated with a second retina (e.g., both eyes sampled concurrently). Additionally, the infrared LED 216 may be co-located with the illumination LED 208, the infrared LED 216 may be swapped in position with the illumination LED 208, or the infrared LED 216 and the illumination LED 208 may be positioned in alignment or differently with respect to the imaging lens arrangement 202. Furthermore, the image sensor 222 may be co-located with the infrared imager 226 and/or have their respective positioned swapped or changed. The arrangement 200 can also be adapted or used for non-retinal, facial, body, eye, or other imagery purposes, such as for any other scientific, research, investigative, or learning purpose.

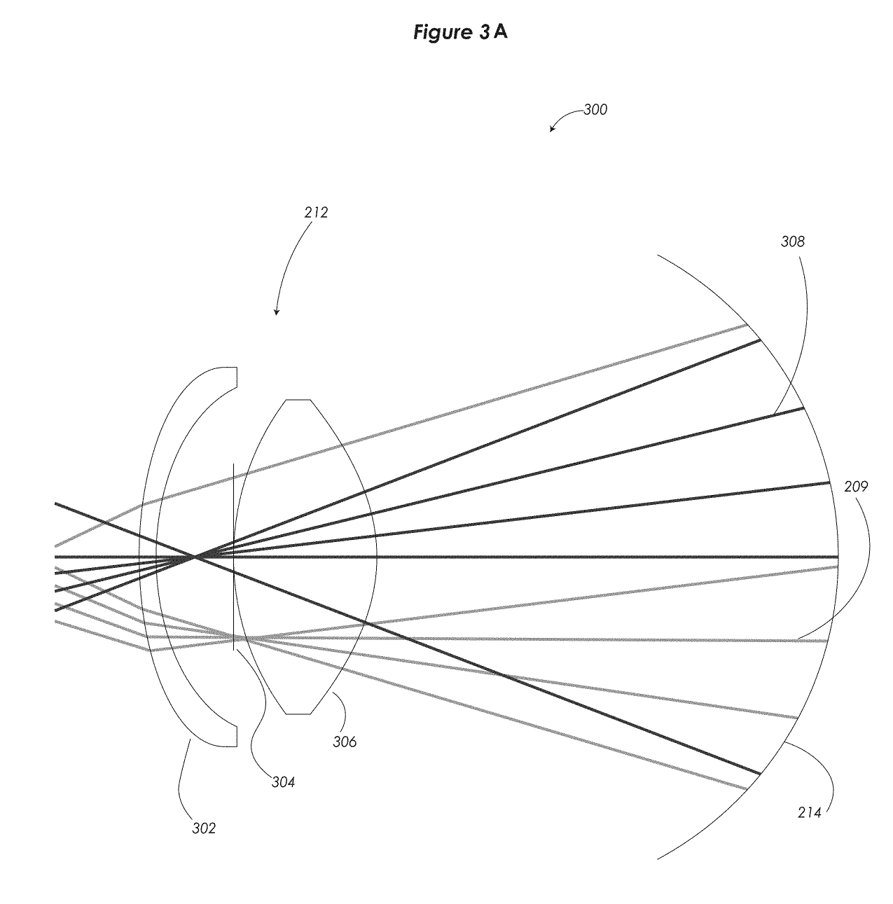

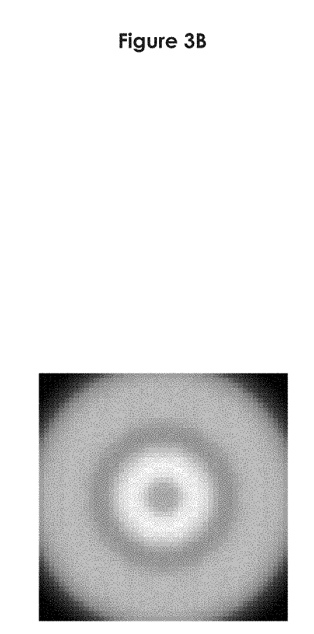

FIG. 3A is a zoom side view of anatomical structures of an eye 300 positioned with a retinal imager device with edge processing, in accordance with an embodiment. The eye 300 can be a left or right eye of an individual and is positioned with the arrangement 200. The eye 300 includes the cornea 302, the pupil 304, the lens 306, and the retina 214. The light rays of FIG. 3A are simplified for illustration and clarity, but in essence the illumination light 209 from the illumination LED 208 enters and passes through the cornea 302, the pupil 304, and the lens 306 before being reflected by the retina 214 as imaging light 308. The illumination light 209 provides annular illumination input to the retina 214. The imaging light 308 is reflected back through the lens 306, the pupil 304, and the cornea 302 for capture by the image sensor 222 as retinal image data with a field of view of approximately forty-two degrees. Due to the positioning of the one or more masks 210, the illumination light 209 and the imaging light 308 have paths that do not intersect or minimally intersect within the scattering elements of the eye (e.g. the lens 306 and the cornea 302). The one or more masks 210 reduce stray light, but can result in non-uniform illumination of the retina that is compensated using one or more compensation program operations (FIG. 3B). In certain embodiment, the one or more masks 210 (and/or the one or more infrared masks 220) can be moved to adjust the illumination light 209 distribution on the retina 214.

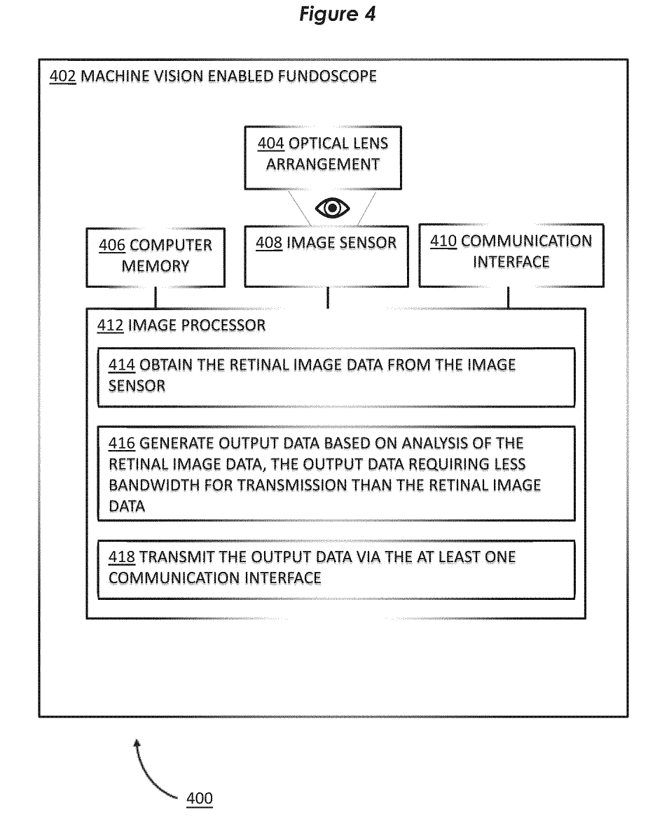

FIG. 4 is a component diagram 400 of a retinal imager device 402 or fundoscope with edge processing, in accordance with an embodiment. In one embodiment, the machine-vision enabled fundoscope 402 for retinal analysis includes, but is not limited to, an optical lens arrangement 404, an image sensor 408 positioned with the optical lens arrangement 404 and configured to convert detected light to retinal image data, computer readable memory 406, at least one communication interface 410, and an image processor 412 communicably linked to the image sensor 408, the computer readable memory 406, and the at least one communication interface 410, the image processor 408 being programmed to execute operations including at least: obtain the retinal image data from the image sensor at 412, generate output data based on analysis of the retinal image data, the output data requiring less bandwidth for transmission than the retinal image data 416, and transmit the output data via the at least one communication interface 418. The retinal imager device 402 or fundoscope can assume the form of the retinal imager device 100 or a different form.

Within the fundoscope 402, the optical lens arrangement 404 is arranged to focus light onto the image sensor 408 as discussed herein. The image sensor 408 is coupled via a high bandwidth link to the image processor 412. The image processor 412 is then coupled to the computer memory 406 and to the communication interface 410 for communication via a communication link having low bandwidth capability.

The optical lens arrangement 404 can include any of the optical arrangements discussed herein, such as arrangement 200, illumination lens arrangement 204, and/or imaging lens arrangement 202, or another different optical arrangement and are directed to a particular field of view associated with a human retina. The optical lens arrangement 404 can be stationary and/or movable, rotatable, pivotable, or slidable.

The image sensor 408 includes a high pixel density imager enabling ultra-high resolution retinal imaging. For instance, the image sensor 408 can include at least an eighteen or twenty-megapixel sensor that provides around twenty gigabytes per second in image data, ten thousand pixels per square degree, and a resolution of at least approximately twenty microns. One particular example of the image sensor 408 is the SONY IMX 230, which includes 5408 H×4412 V pixels of 1.12 microns.

The image sensor 408 is communicably linked with the image processor via a high bandwidth communication link. The relatively high bandwidth communication link enables the image processor 412 to have real-time or near-real-time access to the ultra-high resolution imagery output by the image sensor 408 in the tens of Gbps range. An example of the high bandwidth communication link includes a MIPI-CSI to LEOPARD/INTRINSYC adaptor that provides data and/or power between the image processor 412 and the image sensor 408.

The image processor 412 is communicably linked with the image sensor 408. Due to the high bandwidth communication link, the image processor 412 has full access to every pixel of the image sensor 408 in real-time or near-real-time. Using this access, the image processor 412 performs one or more operations on the full resolution retinal imagery prior to communication of any data via the communication interface 410 (e.g., “edge processing”). Example operations for functions executed by the image processor 412 include, but are not limited to, obtain the retinal image data from the image sensor at 412, generate output data based on analysis of the retinal image data, the output data requiring less bandwidth for transmission than the retinal image data 416, and transmit the output data via the at least one communication interface 418. Other operations and or functions executed by the image processor 412 are discussed and illustrated herein. One particular example of the image processor 412 includes a cellphone-class SOM, such as SNAPDRAGON SOM. The image processor 412 can also be any general purpose computer processor, such as an INTEL or AMTEL computer processor, programmed or configured to perform special purpose operations as disclosed herein.

In certain embodiments, the fundoscope 402 can include a plurality of the optical lens arrangement 404/image sensor 408/image processor 412 combinations linked to a hub processor via a backplane/hub circuit to leverage and distribute processing load. Each of the optical lens arrangements 404 can be directed to an overlapping field of view or a partial segment of the retina, such as to increase an overall resolution of the retinal image data.

The communication interface 410 provides a relatively low bandwidth communication interface between the image processor 412 and a client, device, server, or cloud destination via a communication link on the order of Mbps. While the communication interface 410 may provide the highest wireless bandwidth available or feasible, such bandwidth is relatively low as compared to the high bandwidth communication between the image sensor 408 and the image processor 412 within the fundoscope 402. Thus, the image processor 412 does not necessarily transmit all available pixel data via the wireless communication interface 410, but instead uses edge processing on-board the fundoscope 402 to enable collection of the very high resolution retinal imagery and selection/reduction of that retinal imagery for transmission (or non-transmission) via the communication interface 410. The communication interface 410 can, in certain embodiments, be substituted with a wire-based network interface, such as ethernet, USB, and/or HDMI. One particular example of the communication interface includes a cellular, WIFI, BLUETOOTH, satellite network, and/or websocket enabling communication over the internet with a client running JAVASCRIPT, HTML5, CANVAS GPU, and WEBGL. For instance, an HTML-5 client with a zoom viewer application can connect to an ANDROID server video/camera application of the fundoscope 402 via WIFI to stream retinal imagery at approximately 720p.

The computer memory 406 can include non-transitory computer storage memory and/or transitory computer memory. The computer memory 406 can store program instructions for configuring the image processor 412 and/or store raw retinal image data, processed retinal image data, derived alphanumeric text or binary data, or other similar information.

Example operations and/or characteristics of the fundoscope 402 can include one or more of the following: enable user-self imaging in approximately twenty seconds to three minutes, enable manual or automated capture of retinal images without pupil dilation (non-mydriatic), provide automatic alignment, capture a wide angle retinal image of approximately forty plus degrees, enable adjustable focus, enable multiple image capture of high resolution retinal imagery per session, enable display/review of captured retinal imagery, transmit high resolution retinal imagery in real-time or in batch or at intervals using relatively low bandwidth communication links (e.g., 1-2 Mbps) (e.g., from satellite to ground station), enable self-testing, perform automated image comparison or analysis of images, detect differences in retinal images such as between a current image vs. baseline image, detect a health issue, reduce to text output, perform machine vision or on-board/in-situ/edge processing, enable remote viewing of high resolution imagery using standard relatively low bandwidth communication links (e.g., wireless or internet speeds), enable monitoring of patients remotely and as frequently as needed, detect diabetic retinopathy, macular degeneration, cardiovascular disease, glaucoma, malarial retinopathy, Alzheimer's disease via on-site/on-board/edge processing, transmit a video preview of the zoom-able window to a client computer or device to enable browsing of high resolution retinal imagery, enable transmission of full resolution imagery to a client device or computer for the field of view and zoom level requested, and/or enable machine vision applications or 3^{rd} party applications.

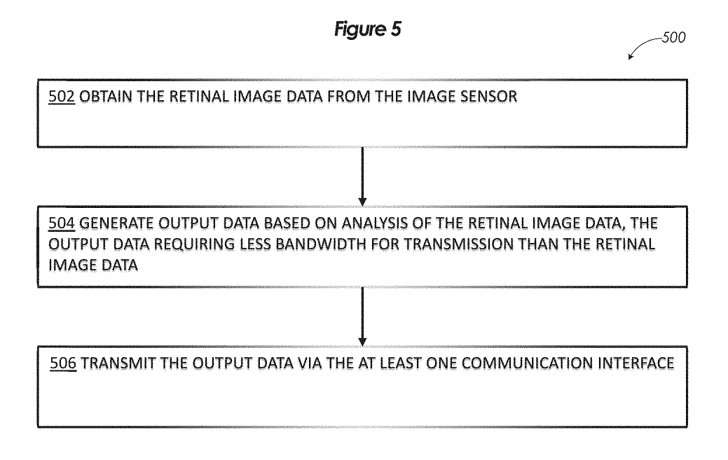

FIG. 5 is a block diagram of a process 500 implemented using a retinal imager device 400 with edge processing, in accordance with various embodiments. In one embodiment, process 500 is executed by a computer processor component 412 of a fundoscope 402 that includes an optical lens arrangement 404, an image sensor 408 configured to convert detected light to retinal image data, and at least one communication interface 410, the process including at least obtain the retinal image data from the image sensor at 502, generate output data based on analysis of the retinal image data, the output data requiring less bandwidth for transmission than the retinal image data at 504, and transmit the output data via the at least one communication interface at 506.

For example, the processor 412 can obtain ultra-high resolution retinal imagery from the image sensor 408 and can select a wide field of view and low zoom of the retinal imagery. Due to the very high resolution of the retinal image data, the processor 412 can decimate pixels within the selected field of view to reduce the image data to a still relatively high-resolution for transmission to a client device via the communication interface 410. The pixel decimation results in lower bandwidth requirements for transmission, but the transmitted retinal image data may still meet or exceed the resolution capabilities of a display screen of the client device.

As an additional example, the processor 412 can obtain ultra-high resolution retinal imagery from the image sensor 408 and can select a narrow field of view and high zoom of the retinal imagery. Due to the very high resolution of the retinal image data, the processor 412 can decimate few to no pixels within the selected field of view and decimate many to all pixels outside the selected field of view to reduce the image data and maintain a high resolution and high acuity for transmission to a client device via the communication interface 410. The selective pixel decimation results in lower bandwidth requirements for transmission, but the transmitted retinal image data provides high acuity for the portion of the selected field of view on a display screen of the client device.

As a further example, the processor 412 can obtain ultra-high resolution retinal imagery from the image sensor 408 and compare the obtained retinal imagery to stored historical or baseline retinal imagery to detect one or more pathologies. In an event no pathologies are detected, the processor 412 can transmit no image data or, in certain embodiments, transmit a binary or alphanumeric text indication of a result of the analysis. The load on the communication interface 410 can thereby be reduced by avoiding image data transmission or transmitting data that requires only a few bytes per second.

As yet a further related example, the processor 412 can obtain ultra-high resolution retinal imagery from the image sensor 408 and compare the obtained retinal imagery to stored historical or baseline retinal imagery to detect one or more pathologies. In an event a potential pathology is detected, the processor 412 can transmit a selected field of view or portion of the retinal image data pertaining to the pathology or, in certain embodiments, transmit a binary or alphanumeric text indication of a result of the analysis. The load on the communication interface 410 can thereby be reduced by tailoring image data for transmission or transmitting data that requires only a few bytes per second.

The foregoing example embodiments are supplemented or expanded herein by many other examples and illustrations of the operations of process 500.

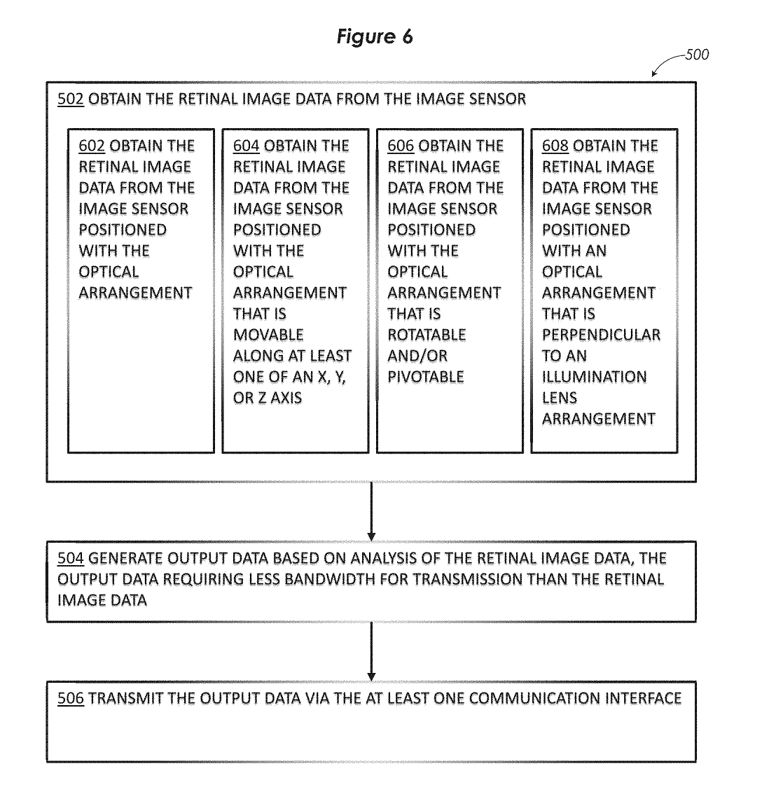

FIG. 6 is a block diagram of a process 500 implemented using a retinal imager device 400 with edge processing, in accordance with various embodiments. In one embodiment, the obtain the retinal image data from the image sensor at 502 includes one or more of obtain the retinal image data from the image sensor positioned with the optical arrangement at 602, obtain the retinal image data from the image sensor positioned with the optical arrangement that is movable along at least one of an x, y, or z axis at 604, obtain the retinal image data from the image sensor positioned with the optical arrangement that is rotatable and/or pivotable at 606, or obtain the retinal image data from the image sensor positioned with an optical arrangement that is perpendicular to an illumination lens arrangement at 608.

In one embodiment, the image processor 412 obtains the retinal image data from the image sensor 408 positioned with the optical arrangement 404 at 602. The image sensor 408 can be positioned with the optical arrangement 404 as illustrated and described with respect to FIGS. 1 and/or 2. However, the image sensor 408 can be positioned in a common axis with the optical arrangement 404, a perpendicular axis with the optical arrangement 404, an obtuse or acute axis with the optical arrangement 404, or some other position relative to the optical arrangement 404. The image sensor 408 can move relative to the optical arrangement 404. Alternatively, one or more lenses of the optical arrangement 404 can move relative to the image sensor 408, such as for focusing light on the image sensor 408. The image sensor 408 can be removable, changeable, and/or replaceable, such as to enable use of image sensors 408 having a variety of characteristics, capabilities, or resolutions.

In one embodiment, the image processor 412 obtains the retinal image data from the image sensor 408 positioned with the optical arrangement 404 that is movable along at least one of an x, y, or z axis at 604. The optical arrangement 404 can move in various directions in order, for example, to accommodate a position of an eye of a user. That is, the optical arrangement 404 can be moved up, back, down, forward, left, or right to be in a position where an eyepiece coincides with a position of an eye of a particular user (e.g., automatic detection of eye position and movement of the optical arrangement or housing containing the optical arrangement to move the eyepiece to the eye position). Alternatively, the optical arrangement 404 can be moved to a particular position that corresponds to an average height, location, and/or position of an eye for various individuals. Additionally, the optical arrangement 404 can be moved manually or automatically between eyes of an individual (e.g., left and right) during a sampling session, such that the individual maintains a constant position with respect to any eyepiece or eyebox during the sampling session. In these examples, the optical arrangement 404 can move or a housing containing the optical arrangement 404 can move.

In one embodiment, the image processor 412 obtains the retinal image data from the image sensor 408 positioned with the optical arrangement 404 that is rotatable and/or pivotable at 606. For example, the optical arrangement 404 can rotate relative to a support structure, such as a table, post, or extension to enable retinal image sampling from different positions. Additionally, the optical arrangement 404 can move along a curve, such as to track a head shape or eye position of a particular user. This can occur during retinal image sampling, such as to obtain different angles of image data while one or more eyes of an individual remain stationary. The rotation, pivoting, or movement of the optical arrangement 404 can be manual or automatic, such as through use of an electromagnetic motor. Furthermore, the optical arrangement 404 can rotate, pivot, or move or a housing containing the optical arrangement 404 can rotate, pivot, or move.

In one embodiment, the image processor 412 obtains the retinal image data from the image sensor 408 positioned with an optical arrangement 404 that is perpendicular to an illumination lens arrangement at 608. For example, FIG. 2 illustrates an illumination lens arrangement 204 that is perpendicular to an imaging lens arrangement 202, whereby the illumination lens arrangement 204 directs illumination light 209 into the imaging lens arrangement 202 using the polarizing splitter/combiner 206. Through the use of one or more masks 210, a path of the illumination light 209 can be controlled to reduce or eliminate intersection with a path of imaging light 308 within the scattering elements of the eye 212 as depicted in FIG. 3A. The image sensor 408 can alternatively be positioned with an optical arrangement 404 that is other than perpendicular to an illumination lens arrangement. For instance, the optical arrangement 404 can be obtuse, orthogonal, acute, or movable relative to an illumination lens arrangement. In certain circumstances, the illumination lens arrangement is omitted.

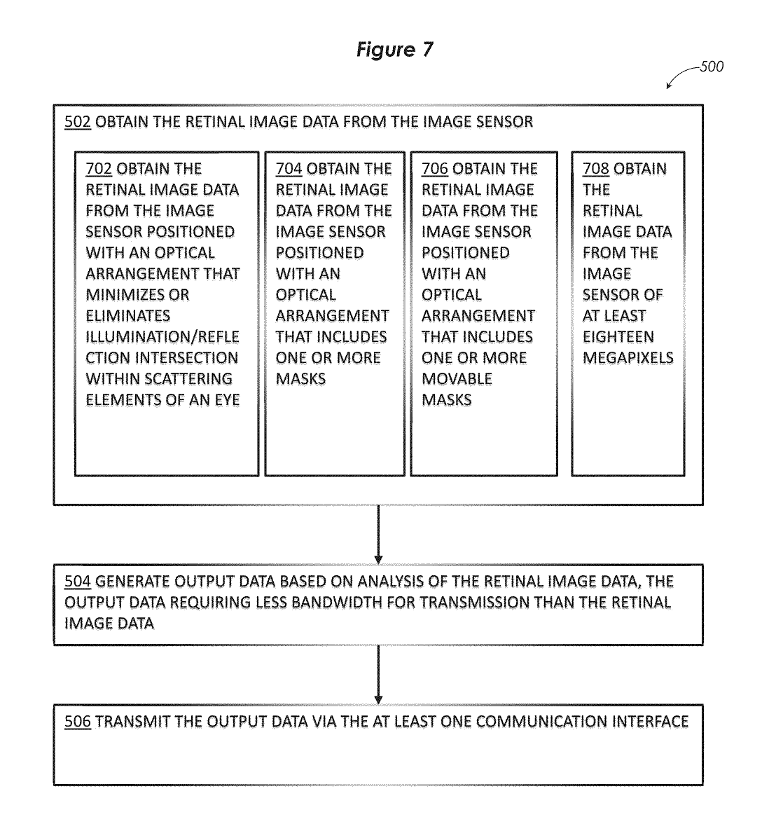

FIG. 7 is a block diagram of a process 500 implemented using a retinal imager device 400 with edge processing, in accordance with various embodiments. In one embodiment, the obtain the retinal image data from the image sensor at 502 includes, but is not limited to, obtain the retinal image data from the image sensor positioned with an optical arrangement that minimizes or eliminates illumination/reflection intersection within scattering elements of an eye at 702, obtain the retinal image data from the image sensor positioned with an optical arrangement that includes one or more masks at 704, obtain the retinal image data from the image sensor positioned with an optical arrangement that includes one or more movable masks at 706, or obtain the retinal image data from the image sensor of at least eighteen megapixels at 708.

In one embodiment, the image processor 412 obtains the retinal image data from the image sensor 408 positioned with an optical arrangement 404 that minimizes or eliminates illumination/reflection intersection within scattering elements of an eye at 702. FIG. 3A illustrates the scattering elements 212 of the eye, including the cornea 302 and the lens 306, which focus and/or scatter incoming light against the retina 214. Illumination light 209 is directed along a path through the scattering elements of the eye 212 and distributed against one or more portions of the retina 214. Some of the illumination light 209 is reflected as the imaging light 308 which passes along a path back through the scattering elements of the eye 212 for detection. The optical arrangement 404 is configured to minimize the interaction and/or interference of the illumination light 209 and the reflected imaging light 308 within or in an area proximate to the scattering elements of the eye 212.

In one embodiment, the image processor 412 obtains the retinal image data from the image sensor 408 positioned with an optical arrangement 404 that includes one or more masks at 704 or obtains the retinal image data from the image sensor 408 positioned with an optical arrangement 404 that includes one or more movable masks at 706. FIG. 2 illustrates the one or more masks 210 positioned proximate to the illumination LED 208. Light 209 from the illumination LED 208 passes to and is at least partially obscured by the one or more masks 210 before passing through the illumination lens arrangement 204 and into the imaging lens arrangement 202. The light 209 is then directed to the retina 214. The position of the one or more masks 210 therefore affects a path of the light 209 from the illumination LED 208, the location of the light 209 within the scattering elements 212 of the eye, and ultimately an area of illumination at the retina 214. In certain circumstances, the one or more masks 210 includes anywhere from one to three or more masks 210. The one or more masks 210 can be positioned at one point along a path of the light 209 or at different points sequentially along a path of the light 209. The one or more masks 210 can be total or partial obscuring masks, such as masks that obscure a percentage of total the light 209, masks that polarize the light 209, or masks that filter the light 209. In one particular embodiment, the one or more mask 210 are movable, such as manually or automatically, to adjust a path of the light 209 or an area of illumination on the retina 214. For example, the one or more masks 210 can be automatically moved to illuminate various portions of the retina 214 and resultant retinal image data can be stitched together to establish a comprehensive retinal image view.

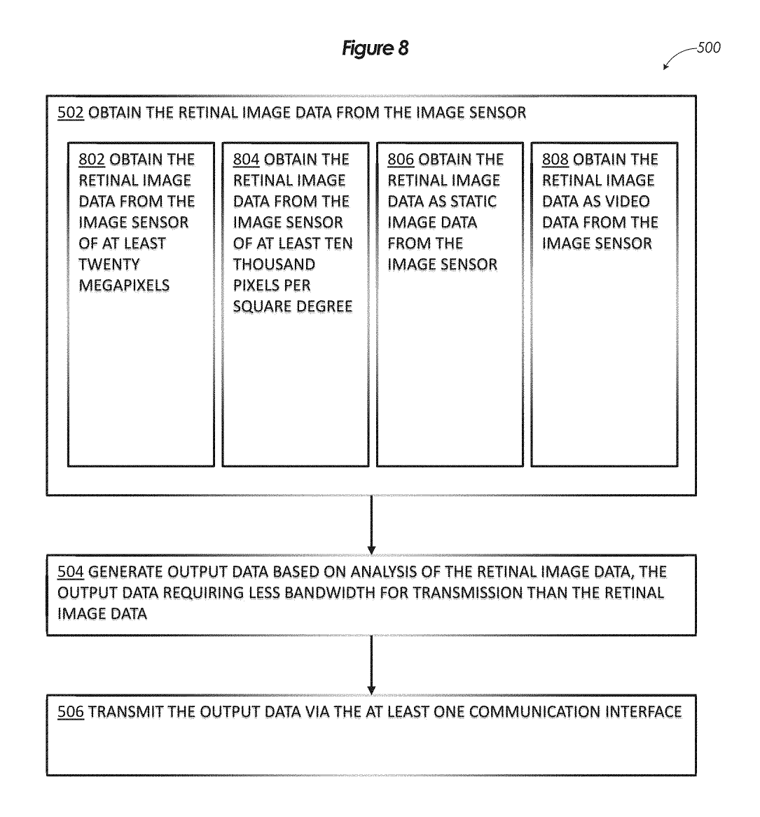

FIG. 8 is a block diagram of a process 500 implemented using a retinal imager device 400 with edge processing, in accordance with various embodiments. In one embodiment, the obtain the retinal image data from the image sensor at 502 includes one or more of obtain the retinal image data from the image sensor of at least twenty megapixels at 802, obtain the retinal image data from the image sensor of at least ten thousand pixels per square degree at 804, obtain the retinal image data as static image data from the image sensor at 806, or obtain the retinal image data as video data from the image sensor at 808.

In one embodiment, the image processor 412 obtains the retinal image data from the image sensor 408 of at least eighteen megapixels at 708 or twenty megapixels at 802. The image sensor 408 provides ultra-high resolution imagery, which can range from approximately one megapixel to around twenty megapixels to a hundred or more megapixels. In certain embodiments, the image sensor 408 contains the highest number of pixels technologically/commercially available. The image sensor 408 therefore enables capture of retinal image data with an extremely high level of resolution and visual acuity. The image processor 412 has access to the full resolution retinal imagery captured by the image sensor 408 for analysis, field of view selection, focus selection, pixel decimation, resolution reduction, static object removal, unchanged object removal, or other operation illustrated or disclosed herein.

In one embodiment, the image processor 412 obtains the retinal image data from the image sensor 408 of at least ten thousand pixels per square degree at 804. As discussed, the image sensor 408 provides ultra-high resolution imagery, which can range from approximately one a thousand pixels per square degree to tens of thousands of pixels per square degree. In certain embodiments, the image sensor 408 contains the highest number of pixels technologically/commercially available. In certain other embodiments, the pixel density varies or is non-uniform in distribution across the image sensor 408 to provide greater resolution for certain retinal areas as compared to other retinal areas. Note that the pixel density can be measured in square inches or square centimeters or by some other metric. In any case the image sensor 408 therefore enables capture of retinal image data with an extremely high level of resolution and visual acuity. The image processor 412 has access to the full resolution retinal imagery captured by the image sensor 408 for analysis, field of view selection, focus selection, pixel decimation, resolution reduction, static object removal, unchanged object removal, or other operation illustrated or disclosed herein.

In one embodiment, the image processor 412 obtains the retinal image data as static image data from the image sensor 408 at 806. Thus, the image processor can obtain one or more retinal images as static image data at one or more different times, triggered by a manual indication or automatic indication such as by control from a computer program. The static retinal image data can be associated with an entire field of view or of a select field of view of the retina. For instance, the static retinal image data can include a series of images each covering a portion of the retina, with illumination and/or masks changing between each of the images. Alternatively, the static retinal image data can include a sequence of images covering overlapping fields of view, which may be used for resolution enhancement and/or stitching. Additionally, the static retinal image data can include retinal images for left and right eyes of an individual.

In one embodiment, the image processor 412 obtains the retinal image data as video data from the image sensor 408 at 808. Thus, the image processor 412 can obtain one or more retinal videos comprised of a series of static images over one or more time periods (e.g., approximately twenty frames per second). The collection of the one or more retinal videos may be triggered by a manual indication or automatic indication such as by control from a computer program. The retinal video data can be associated with an entire field of view or of a select field of view of the retina. For instance, the retinal video data can include digitally recreated movement or panning over various portions of the retina, with illumination and/or masks changing during the movement or panning. Additionally, the retinal video data can include retinal videos for left and right eyes of an individual.

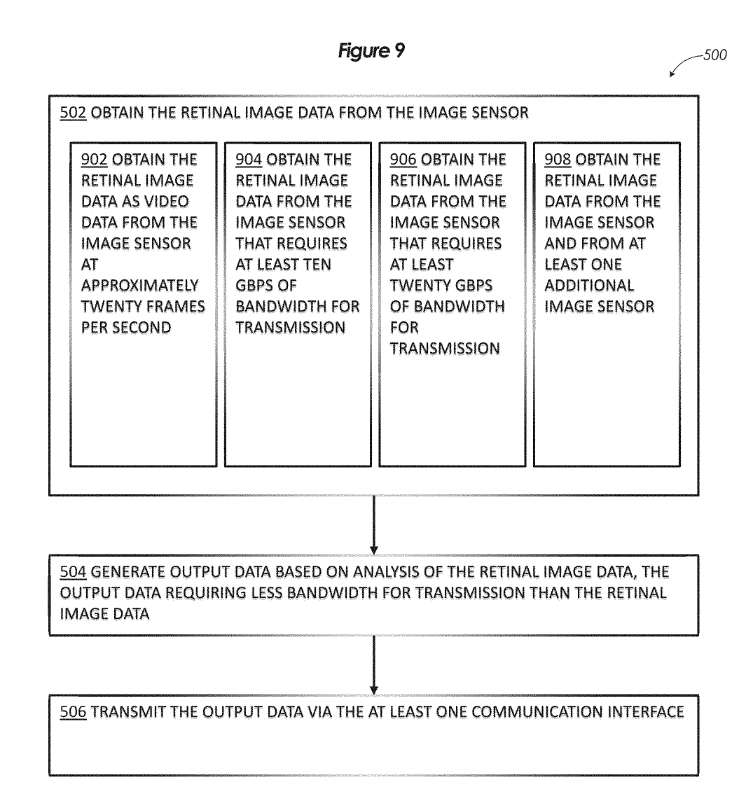

FIG. 9 is a block diagram of a process 500 implemented using a retinal imager device 400 with edge processing, in accordance with various embodiments. In one embodiment, the obtain the retinal image data from the image sensor at 502 includes one or more of obtain the retinal image data as video data from the image sensor at approximately twenty frames per second at 902, obtain the retinal image data from the image sensor that requires at least ten Gbps of bandwidth for transmission at 904, obtain the retinal image data from the image sensor that requires at least twenty Gbps of bandwidth for transmission at 906, or obtain the retinal image data from the image sensor and from at least one additional image sensor at 908.

In one embodiment, the image processor 412 obtains the retinal image data as video data from the image sensor 408 at approximately twenty frames per second at 902. The frame rate of the video data can be more or less than twenty frames per second depending upon a particular application. For instance, the frame rate can be slowed to approximately one frame per second or can be increased to approximately thirty or more frames per second. The frame rate can be adjustable based on user input or an application control. In certain embodiments, multiple frames from the video data are usable to generate an enhanced resolution static image by combining pixels from the multiple frames of video data.

In one embodiment, the image processor 412 obtains the retinal image data from the image sensor 408 that requires at least ten Gbps of bandwidth for transmission at 904 or at least twenty Gbps of bandwidth for transmission at 906. As discussed herein, the image sensor 408 has high resolution pixel density. Whether the image processor 412 retains the retinal image data from the image sensor 408 in a form of static image data or video image data, the amount of captured imagery is significant and can be on the order of ten, twenty, or more gigabytes per second. This volume of image data is incapable of being timely transmitted in its entirety via a communication interface that can be limited to a few megabytes per second (e.g., wireless communication interface). Thus, operations disclosed herein are performed by the image processor 412 on-board or at-the-edge with the fundoscope 402 prior to any transmission of the image data. Thus, the image processor 412 has high bandwidth access to full resolution imagery captured by the image sensor 408 to perform analysis, pathology detection, imagery comparisons, selective pixel decimation, selective pixel retention, static imagery removal, or other operations discussed herein. The output of the image processor 412 following any full-resolution processing operations can require less bandwidth and may be more timely transmittable via the communication interface 410.

In one embodiment, the image processor 412 obtains the retinal image data from the image sensor 408 and from at least one additional image sensor at 908. For example, the at least one additional image sensor can be associated with an additional lens arrangement, whereby each of the image sensor 408 and the at least one additional image sensor capture image data associated with different segments of the retina, with overlapping portions of the retina, or with different retinas (e.g., left and right retinas of an individual sampled substantially concurrently or sequentially). Alternatively, the at least one additional image sensor can be an infrared image sensor configured to capture infrared image data, which is usable by the image processor 412 to perform functions such as focus and eye positioning or centering while avoiding an iris constriction response.

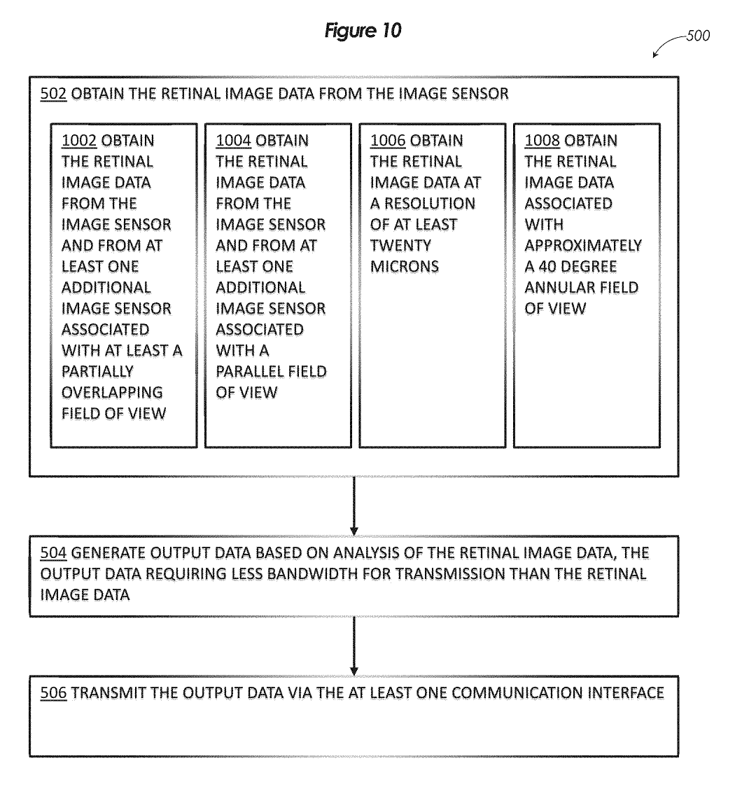

FIG. 10 is a block diagram of a process 500 implemented using a retinal imager device 400 with edge processing, in accordance with various embodiments. In one embodiment, the obtain the retinal image data from the image sensor at 502 includes one or more of obtain the retinal image data from the image sensor and from at least one additional image sensor associated with at least a partially overlapping field of view at 1002, obtain the retinal image data from the image sensor and from at least one additional image sensor associated with a parallel field of view at 1004, obtain the retinal image data at a resolution of at least twenty microns at 1006, or obtain the retinal image data associated with approximately a 40 degree annular field of view at 1008.

In one embodiment, the image processor 412 obtains the retinal image data from the image sensor 408 and from at least one additional image sensor associated with at least a partially overlapping field of view at 1002 or from at least one additional image sensor associated with a parallel field of view at 1004. Each of the image sensors can capture ultra-high resolution imagery, which can be independently analyzed or combined by the image processor 412. For instance, one image sensor can capture left retina image data and another image sensor can capture right retina image data. Independent image processors can simultaneously process the respective left and right retina image data and perform functions and operations disclosed herein, such as retinal analysis, pathology detection, change detection, pixel decimation, pixel selection, unchanged pixel removal, or other operation. Concurrent processing of the left and right retina image data can reduce the duration of overall retinal analysis and testing.

In one embodiment, the image processor 412 obtains the retinal image data at a resolution of at least twenty microns at 1006. The retinal image data can have a resolution of hundreds or thousands of microns or can have a resolution as detailed as low as ten or less microns. Various optical arrangements 404 and/or image sensors 408 can be used limited only to that technologically and commercially available or limited to that permitted by budget or need. Approximately twenty microns is sufficient in some embodiments to provide ultra-high visual acuity of a retina to enable the image processor to perform the various operations and functions disclosed and illustrated herein.

In one embodiment, the processor 412 obtains the retinal image data associated with approximately a forty-degree annular field of view at 1008. The optical lens arrangement 404 can include the imaging lens arrangement 202 illustrated in FIG. 2, which provides for approximately a +/−21.7 degree field of view from center. However, different fields of view are possible with different lens arrangements, from very narrow fields of view of approximately a few degrees to very broad fields of view of more than forty degrees. In certain embodiments, the optical arrangement can be configured to provide an adjustable, modifiable, or selectable field of view. In other embodiments, the optical arrangement 404 can be replaceable with a different optical arrangement to achieve a different field of view.

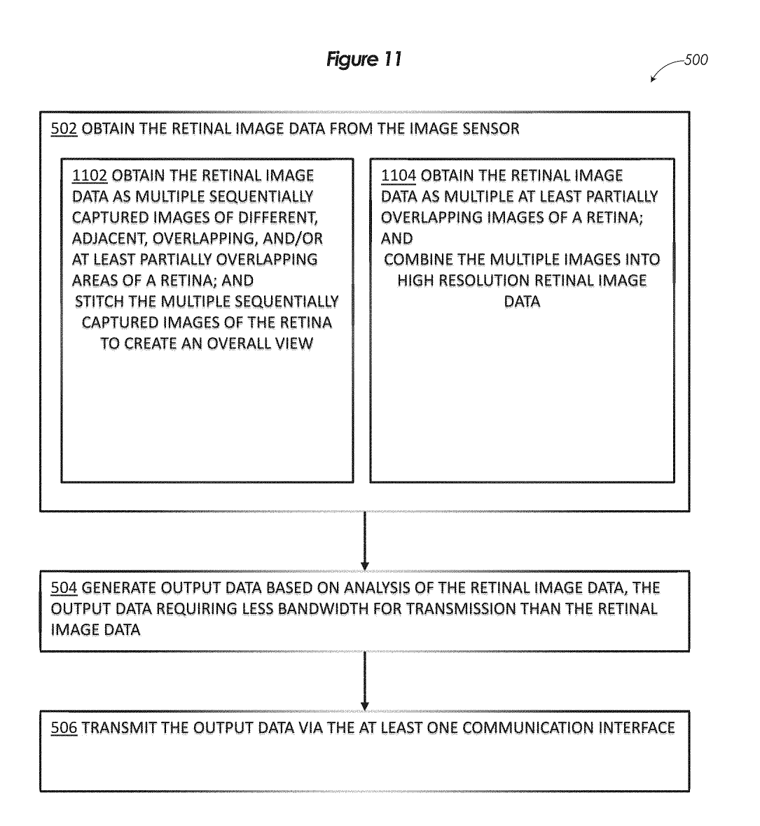

FIG. 11 is a block diagram of a process 500 implemented using a retinal imager device 400 with edge processing, in accordance with various embodiments. In one embodiment, the obtain the retinal image data from the image sensor at 502 includes one or more of obtain the retinal image data as multiple sequentially captured images of different, adjacent, overlapping, and/or at least partially overlapping areas of a retina and stitch the multiple sequentially captured images of the retina to create an overall view at 1102 and/or obtain the retinal image data as multiple at least partially overlapping images of a retina and combine the multiple images into high resolution retinal image data at 1104.

In one embodiment, the image processor 412 obtains the retinal image data as multiple sequentially captured images of different, adjacent, overlapping, and/or at least partially overlapping areas of a retina and stitches the multiple sequentially captured images of the retina to create an overall view at 1102. For instance, the image processor 412 can obtain from the image sensor 408 retinal image data of a left-bottom quadrant, a left-top quadrant, a right-top quadrant, and a right-bottom quadrant associated with a retina, each with approximately a five percent overlap with adjacent quadrant images. The image processor 412 can stitch the quadrant images together using the overlapping portions for positional alignment to create an overall composite image of the retina. The image processor 412 can obtain fewer or greater number segment images to establish a partial or complete image of the retina. In certain embodiments, the image processor 412 can control illumination changes between obtaining each of the quadrant images of the retina (e.g., through controlled movement of one or more masks associated with an illumination source). In one particular embodiment, the image processor 412 obtains a section or segment retinal image by obtaining imagery for an overall field of view and decimating pixels associated with certain non-selected areas. In another embodiment, the image processor 412 obtains a portion of the retinal imagery by movement or adjustment of the optical lens arrangement 404.

In one embodiment, the image processor 412 obtains the retinal image data as multiple at least partially overlapping images of a retina and combines the multiple images into high resolution retinal image data at 1104. For instance, the image processor 412 can obtain from the image sensor 408 a series of high-resolution retinal images of the same overall view of a retina. The processor 412 can then combine the series of images by adding together at least some of the pixels to increase the pixel density, resolution, and/or visual acuity over any single one of the individual retinal images obtained. In some embodiments, the combination of pixels from multiple retinal images may be uniform or non-uniform. For example, the processor 412 can increase the pixel density for a particular retinal region of interest (e.g., a region that has changed or is exhibiting a particular pathology) while maintaining the pixel density for other areas. Thus, the processor 412 can initiate pixel density enhancements based on one or more trigger events in one or more obtained retinal images, such as detection of a potential problem area, in anticipation of that particular area being requested by a healthcare person.

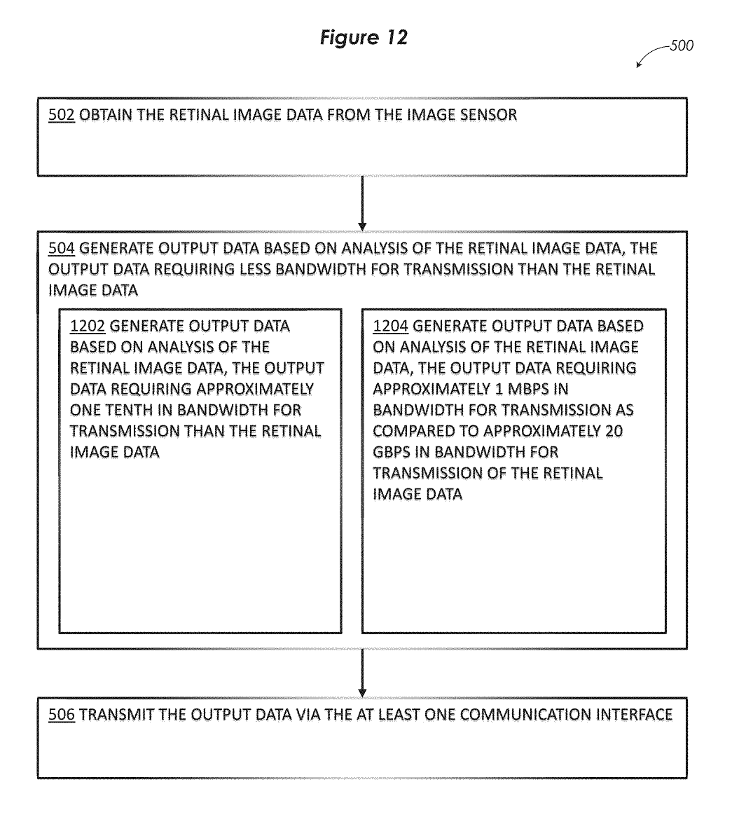

FIG. 12 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data based on analysis of the retinal image data, the output data requiring approximately one tenth in bandwidth for transmission than the retinal image data at 1202, or generate output data based on analysis of the retinal image data, the output data requiring approximately 1 Mbps in bandwidth for transmission as compared to approximately 20 Gbps in bandwidth for transmission of the retinal image data at 1204.

In one embodiment, the image processor 412 generates output data based on analysis of the retinal image data, the output data requiring approximately one tenth in bandwidth for transmission than the retinal image data at 1202 or generates output data based on analysis of the retinal image data, the output data requiring approximately 1 Mbps in bandwidth for transmission as compared to approximately 20 Gbps in bandwidth for transmission of the retinal image data at 1204. The image processor 412 obtains ultra-high resolution imagery from the image sensor 408 for one or more instances in time (e.g., static imagery or video). The volume of raw retinal image data obtained can far exceed the communication bandwidth capabilities of the communication interface 410. For instance, the required bandwidth for communicating all of the raw retinal image data can be ten, twenty, or more times the amount of available bandwidth of the communication interface 410. The processor 412 overcomes this potential deficiency by performing operations on the ultra-high resolution retinal imagery at the fundoscope 402 level, which can be referred to as edge-processing, in-situ-processing, or on-board processing. By performing edge processing of the raw retinal image data, the image processor 412 has access to real-time or near-real time imagery of ultra-high resolution and can generate output data that is reduced in size and/or tailored to a specific need or request. The output data can be significantly less in size for transmission over the communication interface 410, yet be focused, highly-useful, and even of high-resolution/acuity for a particular application or request.

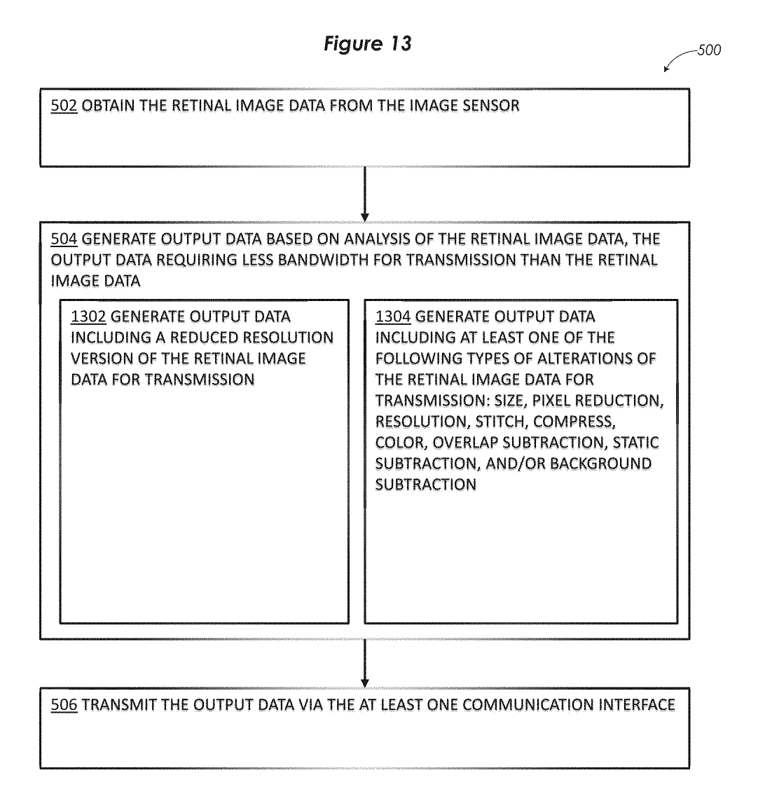

FIG. 13 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data including a reduced resolution version of the retinal image data for transmission at 1302 and/or generate output data including at least one of the following types of alterations of the retinal image data for transmission: size, pixel reduction, resolution, stitch, compress, color, overlap subtraction, static subtraction, and/or background subtraction at 1304.

In one embodiment, the image processor 412 generates output data including a reduced resolution version of the retinal image data for transmission at 1302. The image processor 412 obtains ultra-high resolution imagery from the image sensor 408, which includes a very large number of pixels. The raw retinal imagery may therefore have an overall resolution that far exceeds a screen resolution of a requesting device (e.g. twenty megapixels of raw retinal image data vs. one megapixel display screen). Therefore, the image processor 412 can reduce a resolution of the raw retinal image data to a still very high-resolution that meets or exceeds a display screen resolution of a requesting device or an average display screen resolution. This process can be referred to as pixel decimation and the image processor 412 can perform the pixel decimation uniformly or non-uniformly throughout the retinal image data. The amount of pixel decimation performed by the image processor 412 can also vary by an area of the retinal image data selected. For instance, for a large area of the retinal image data, the image processor 412 can be configured to decimate a larger number of pixels. For a small area (e.g., corresponding to a digital zoom), the image processor 412 can be configured to decimate a smaller to no number of pixels. The variable pixel decimation dependent upon area enables the transmission of constant acuity or constant resolution retinal images.

In one embodiment, the image processor 412 generates output data including at least one of the following alterations of the retinal image data for transmission: size, pixel reduction, resolution, stitch, compress, color, overlap subtraction, static subtraction, and/or background subtraction at 1304. The image processor 412 need not transmit all of the raw retinal image data and can utilize various operations to reduce that raw retinal image data into highly useful data that is focused and targeted. For instance, the image processor 412 can reduce an overall area size of the retinal image data by decimating pixel data other than a particular region of possible interest. Additionally, the image processor 412 can perform pixel decimation or pixel reduction within a selected area of interest to reduce a resolution to a still high resolution for a particular application (e.g., print, large high-definition monitor, mobile phone display, etc). The image processor 412 can, in some embodiments, stitch together various retinal image segments to produce an overall retinal image before performing additional analysis or reduction operations of the overall retinal image. In certain situations, the image processor 412 can identify redundant over overlapping portions of the retinal image data that is requested by multiple users and transmit the redundant or overlapping portions of the retinal image data only once. In some embodiments, the image processor 412 identifies areas of the retinal image data that have not changed since a previous transmission and then removes those areas from transmission, such that a server or client device gap-fills the omitted areas back into the retinal image data. Alternatively, the image processor can transmit a selected portion of the retinal image data at a first resolution and transmit an adjacent area or background portion of the retinal image data at a second resolution that is lower than the first resolution. In this example, the first resolution may be a high resolution relative to a screen display resolution and the second resolution may be a low resolution relative to the screen display. In addition to these operations, the image processor 412 can perform image compression on any image data prior to transmission. Examples of compression techniques performed by the image processor 412 include one or more of reducing color space, chroma subsampling, transform coding, fractal compression, run-length encoding, DPCM, entropy encoding, deflation, chain coding, or the like.

An example operation sequence of the image processor 412 illustrates how one or more of the foregoing techniques can be utilized by the image processor 412. The image processor 412 can obtain the ultra-high resolution retinal imagery from the image sensor 408 and select an overall field of view of substantially the entire area of the retinal imagery. The image processor can identify an area of change (e.g., due to a new manifestation of a pathology). The image processor 412 then performs pixel decimation uniformly across the retinal imagery to reduce the resolution of the retinal imagery to retain approximately 1/10^{th} of the retinal image data. The image processor 412 then further reduces a resolution of the retinal imagery data corresponding to other than the area of change by another fifty percent. The remaining image data is then compressed by the image processor 412 and transmitted within the bandwidth constraints of the communication interface 410 to a client device associated with a physician. The client device is then able to decompress the retinal image data and output the same for display, such that the retinal image data includes high-resolution and low-resolution portions corresponding to the area of change and non-changed areas, respectively. A request received by the image processor 412 for higher resolution imagery associated with non-changed areas can be satisfied then by transmitting via the communication interface 410 only the additional fifty percent of the pixel data for that particular requested area.

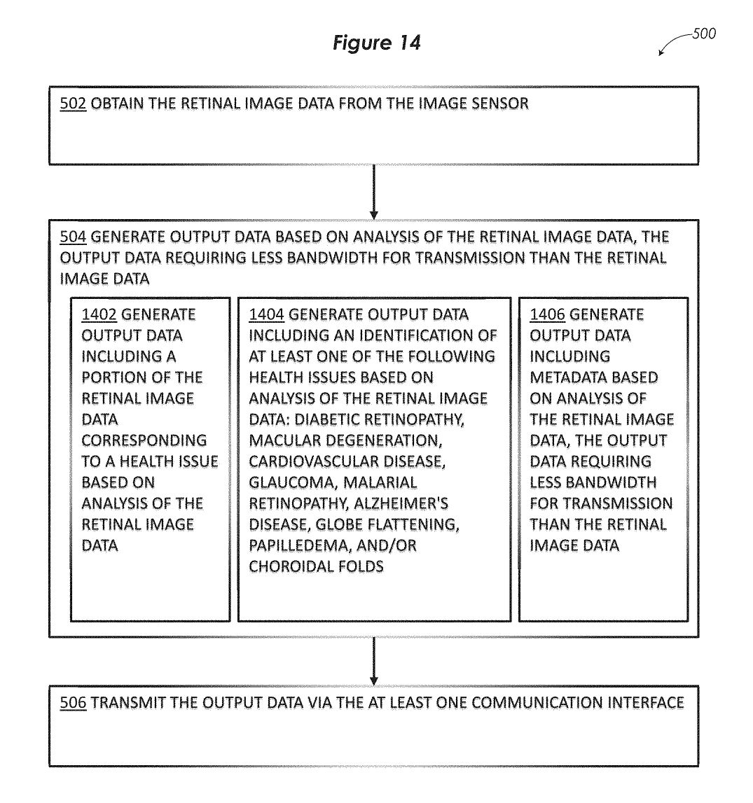

FIG. 14 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data including a portion of the retinal image data corresponding to a health issue based on analysis of the retinal image data at 1402, generate output data including an identification of at least one of the following health issues based on analysis of the retinal image data: diabetic retinopathy, macular degeneration, cardiovascular disease, glaucoma, malarial retinopathy, Alzheimer's disease, globe flattening, papilledema, and/or choroidal folds at 1404, or generate output data including metadata based on analysis of the retinal image data, the output data requiring less bandwidth for transmission than the retinal image data at 1406.

In one embodiment, the image processor 412 generates output data including a portion of the retinal image data corresponding to a health issue based on analysis of the retinal image data at 1402 or generates output data including an identification of at least one of the following health issues based on analysis of the retinal image data: diabetic retinopathy, macular degeneration, cardiovascular disease, glaucoma, malarial retinopathy, Alzheimer's disease, globe flattening, papilledema, and/or choroidal folds at 1404. The image processor 412 has access to ultra-high resolution retinal imagery obtained from the image sensor 408 and can perform image analysis on the retinal imagery prior to any transmission of the retinal imagery on-board, in-situ, and/or using edge processing. The image analysis can include, for example, image recognition analysis and measurements to detect and/or identify one or more potential instances of a pathology. The analysis or measurements performed by the image processor 412 can be based on baseline parameters, changes from previous retinal images of a particular individual, and/or averages for a general or specific patient population. If a retinal pathology is detected or measured, the image processor 412 can generate output data based on the same. The output data generated by the image processor 412 can include a binary indication of the pathology, an alphanumeric description of the pathology or measurements, and/or retinal image data pertaining to the same.

The image processor 412 can be configured to detect and/or measure one or a plurality of various retinal pathologies. For example, the image processor 412 can be configured to detect or measure any one or more of diabetic retinopathy, macular degeneration, cardiovascular disease, glaucoma, malarial retinopathy, Alzheimer's disease, globe flattening, papilledema, and/or choroidal folds. For example, with respect to diabetic retinopathy, the image processor 412 can detect and/or measure in the retina one or more instances of hemorrhages, bleeding, growth of new fragile blood vessels toward the eye center, or blood leakage. With respect to macular degeneration, the image processor 412 can detect and/or measure blood vessel growth, blood leakage, or fluid leakage in the macula area of the retina. With respect to cardiovascular disease, the image processor 412 can detect and/or measure inflammatory markers such as narrower retinal arteriolar diameters or larger retinal venular diameters. With respect to glaucoma, the image processor 412 can detect and/or measure the optic disk, optic cup, and neuroretinal rim and calculate the cup-to-disk ratio and share of the neuroretinal rim. With respect to malarial retinopathy, the image processor 412 can detect and/or measure vessel discoloration, retinal whitening, and hemorrhages or red lesions. With respect to Alzheimer's disease, the image processor 412 can detect and/or measure plaque deposits, venous blood column diameters, or thinning of a retinal nerve fiber layer. With respect to globe flattening, choroidal folds, the image processor 412 can detect and/or measure physical indentation, shape, compression, or displacement in the retina. With respect to papilledema, the image processor 412 can detect and/or measure swelling of the optic disk, engorged or tortuous retinal veins, or retinal hemorrhages around the optic disk. The image processor 412 can be configured to measure or detect any visually detectable parameter including any of the aforementioned or others. Furthermore, the image processor 412 can be configured to have any one or more parameters tied to any one or more potential pathologies. In addition to the listed pathologies, many other pathologies may be detected and/or measured using retinal images, including for example optic disc edema, optic nerve sheath distension, optic disc protraction, cotton wool spots, macular holes, macular puckers, degenerative myopia, lattice degeneration, retinal tears, retinal detachment retinal artery occlusion, branch retinal vein occlusion, central retinal vein occlusion, intraocular tumors, inherited retinal disorders, penetrating ocular trauma, pediatric and neonatal retinal disorders, cytomegalovirus (cmv) retinal infection, macular edema, uveitis, infectious retinitis, central serous retinopathy, retinoblastoma, endophthalmitis, hypertensive retinopathy, retinal hemorrhage, solar retinopathy, retinitis pigmentosa, or other optic nerve or ocular changes.

In one embodiment, the image processor 412 generates output data including metadata based on analysis of the retinal image data, the output data requiring less bandwidth for transmission than the retinal image data at 1406. The metadata generated by the image processor 412 can include a variety of information, such as patient name, time of sampling, age of patient, identified potential pathologies, resolution, frame rate, coordinates of imagery manifesting potential pathologies, measurements, description of pathologies, changes between previous measurements, recommended courses of action, additional physiological measurements (e.g., heart rate, weight, blood pressure, visual acuity of patient, temperature, blood oxygen level, physical activity measurement, skin conductivity), or the like. The metadata can be transmitted with the retinal image data, before any retinal imagery is transmitted, or transmitted without retinal imagery. The metadata can be alphanumeric text, binary, or image data and can therefore require significantly less bandwidth than required for transmission of the high resolution retinal imagery.

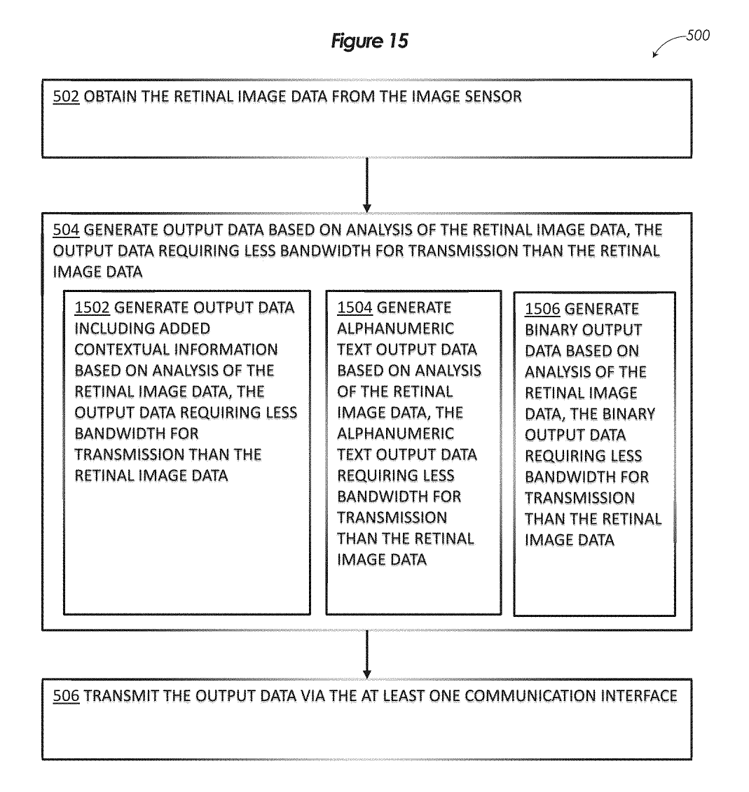

FIG. 15 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data including added contextual information based on analysis of the retinal image data, the output data requiring less bandwidth for transmission than the retinal image data at 1502, generate alphanumeric text output data based on analysis of the retinal image data, the alphanumeric text output data requiring less bandwidth for transmission than the retinal image data at 1504, or generate binary output data based on analysis of the retinal image data, the binary output data requiring less bandwidth for transmission than the retinal image data 1506.

In one embodiment, the image processor 412 generates output data including added contextual information based on analysis of the retinal image data, the output data requiring less bandwidth for transmission than the retinal image data at 1502. For example, the image processor 412 can add information to the retinal image data for transmission via the communication interface 410, such as date/time, subject first/last name, session ID of exam, a highlight indication of the problematic or pathological area (e.g., an arrow or circle added to the image to focus a clinician's attention), or additional historical image data (e.g., past retinal image data of a patient juxtaposed with current retinal image data of the patient to aid in comparisons). The contextual information generated by the image processor 412 can include text, image data, binary data, coordinate information, or the like. The contextual information can be transmitted with retinal image data, before or after retinal image data, or instead or in lieu of retinal image data.

For instance, the image processor 412 can obtain ultra-high resolution retinal imagery from the image sensor 408 and perform image recognition analysis on the retinal imagery to identify one or more instances of hemorrhages, bleeding, growth of new fragile blood vessels toward the eye center, or blood leakage. The image processor can reduce a resolution of the retinal image data to that of an IPHONE 7 display (e.g., 750×1334 pixels) and further reduce a resolution of areas other than those identified by another twenty-five percent. The image processor 412 can then remove all unchanged areas from the image data since a previous transmission and then append contextual information to the retinal image data prior to transmission. The contextual information can include a date, a time, a patient name, and indicia that highlights the identified instances. The image processor 412 then transmits the contextual information with the reduced retinal image data, where the retinal image data is gap-filled with prior transmitted retinal image data prior to forwarding to the IPHONE 7 requesting device.

In one embodiment, the image processor 412 generates alphanumeric text output data based on analysis of the retinal image data, the alphanumeric text output data requiring less bandwidth for transmission than the retinal image data at 1504. The image processor 412 has access to the ultra-high resolution retinal imagery from the image sensor 408. In certain cases, to reduce a bandwidth load on the communication interface 410, the image processor 412 can perform image recognition with respect to the retinal imagery to determine a pathology or lack of pathology and generate alphanumeric text based on the same. For instance, the alphanumeric text can describe a detected pathology or indicate that there is no change since a previous analysis. The alphanumeric text can be a letter, a word, a phrase, or a paragraph, and can include numbers and/or symbols. Thus, the alphanumeric text can be transmitted by the image processor 412 via the communication interface 410, which may only require a few bytes per second in bandwidth as opposed to megabytes per second or gigabytes per second for the raw retinal image data.

For instance, the image processor 412 can obtain the ultra-high resolution imagery from the image sensor 408 and perform image recognition to identify an increase in blood vessel growth, blood leakage, or fluid leakage in the macula area of the retina. The image processor 412 can then generate alphanumeric text such as “Subject John Q. Smith has some indications of macular degeneration in the left eye, including a ten percent increase in blood vessel growth, two instances of blood leakage and/or fluid leakage in the macula of the left retina.”. The image processor 412 can then transmit the alphanumeric text description via the communication interface, requiring only a few bytes per second for transmission, to enable a care provider to consider the same. Retinal image data may be transmitted in response to a request for further information or can be discarded, such as in the event that the care provider is aware of the situation and doesn't need to further review the retinal imagery.

In one embodiment, the image processor 412 generates binary output data based on analysis of the retinal image data, the binary output data requiring less bandwidth for transmission than the retinal image data 1506. The image processor 412 can access the ultra-high resolution retinal imagery from the image sensor 408 and perform image recognition to determine a potential pathology or lack of pathology in the retinal image data. The image processor 412 can then transmit a voltage high or voltage low signal (e.g., 0 or 1), requiring little to no bandwidth, based on the determination. The retinal image data can be transmitted with the binary indication, following the binary indication, or not transmitted depending upon a particular application, request, or program instruction.

For instance, the image processor 412 can perform image recognition or comparative analysis on the ultra-high resolution retinal imagery to determine that there is no change or potential pathology presented. The image processor 412 can then generate a zero indication and transmit the same via the communication interface 410 without requiring any transmission of retinal image data.

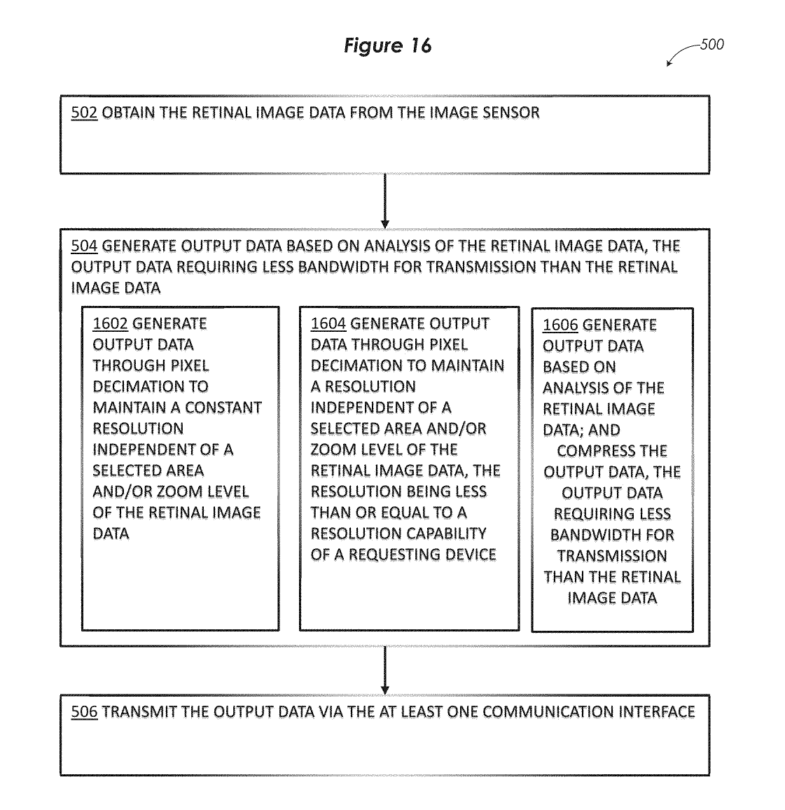

FIG. 16 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data through pixel decimation to maintain a constant resolution independent of a selected area and/or zoom level of the retinal image data at 1602, generate output data through pixel decimation to maintain a resolution independent of a selected area and/or zoom level of the retinal image data, the resolution being less than or equal to a resolution of a client device at 1604, or generate output data based on analysis of the retinal image data and compress the output data, the output data requiring less bandwidth for transmission than the retinal image data at 1606.

In one embodiment, the image processor 412 generates output data through pixel decimation to maintain a constant resolution independent of a selected area and/or zoom level of the retinal image data at 1602. The image processor 412 has access to ultra-high resolution retinal imagery with a very large number of pixels (e.g., twenty or more megapixels). The image processor 412 can decimate pixels of the raw ultra-high resolution retinal imagery to maintain a given resolution (e.g., one to five megapixels). The number of pixels decimated to maintain the given resolution will vary in an inverse relationship to the size of an area/zoom level selected from the raw retinal imagery. That is, the image processor 412 can decimate a large portion of the pixel data when a wide field of view is selected corresponding to substantially the entire retina. This is due to the selection including virtually all of the raw image data and pixels. However, the image processor 412 can decimate few to no pixels when a narrow or small field of view or high zoom level is selected corresponding to a small area of the retina (e.g., the optic nerve or macula area). This is due to the selection including possibly fewer than the given resolution (e.g. fewer than one to five megapixels). In this regard, the image processor can maintain a very high acuity level for wide or low zoom selections through to very small or high zoom selections without substantial difference in the relatively low bandwidth requirement of the communication interface 410.

In one embodiment, the image processor 412 generates output data through pixel decimation to maintain a resolution independent of a selected area and/or zoom level of the retinal image data, the resolution being less than or equal to a resolution of a client device at 1604. The image processor 412 can obtain metadata that indicates a type of requesting device or a screen resolution of the requesting device. Based on the metadata, the image processor 412 can adjust the desired resolution and pixel decimation amounts to provide the highest resolution retinal image data that can be accommodated by a particular device. Thus, for higher screen resolution devices or print applications, for example, the image processor 412 can adjust the decimation amount downward, such that fewer pixels are decimated and a higher resolution image is transmitted. Likewise, for lower screen resolution devices, the image processor 412 can adjust the decimation amount upward, such that more pixels are decimated and a lower resolution image is transmitted. The image processor 412 can adjust the decimation amounts in real-time for various user-requests to accommodate many different devices or applications of the retinal image data.

For instance, the image processor 412 can receive a request from a fourth generation IPAD device with a specified screen resolution of 2048×1536. The image processor can adjust the decimation to maintain approximately a three megapixel resolution for various fields of view and/or zoom selections. The image processor 412 can receive another request from an IWATCH with a specified resolution of 312×390. The image processor can adjust the decimation further in this instance to maintain approximately a 0.1 megapixel resolution for various fields of view and/or zoom selections. In this regard, the image processor 412 provides retinal image data at high resolutions for particular devices while minimizing the bandwidth requirement of the communication interface 410.

In one embodiment, the image processor 412 generates output data based on analysis of the retinal image data and compresses the output data, the output data requiring less bandwidth for transmission than the retinal image data at 1606. The image processor 412 can compress raw retinal image data or compress retinal image data post-reduction (e.g., pixel reduction, static object omission, unchanged area omission, etc). The compressed or coded output data can be transmitted via the communication interface 410 with less bandwidth load. Examples of compression techniques performed by the image processor 412 include one or more of reducing color space, chroma subsampling, transform coding, fractal compression, run-length encoding, DPCM, entropy encoding, deflation, chain coding, or the like.

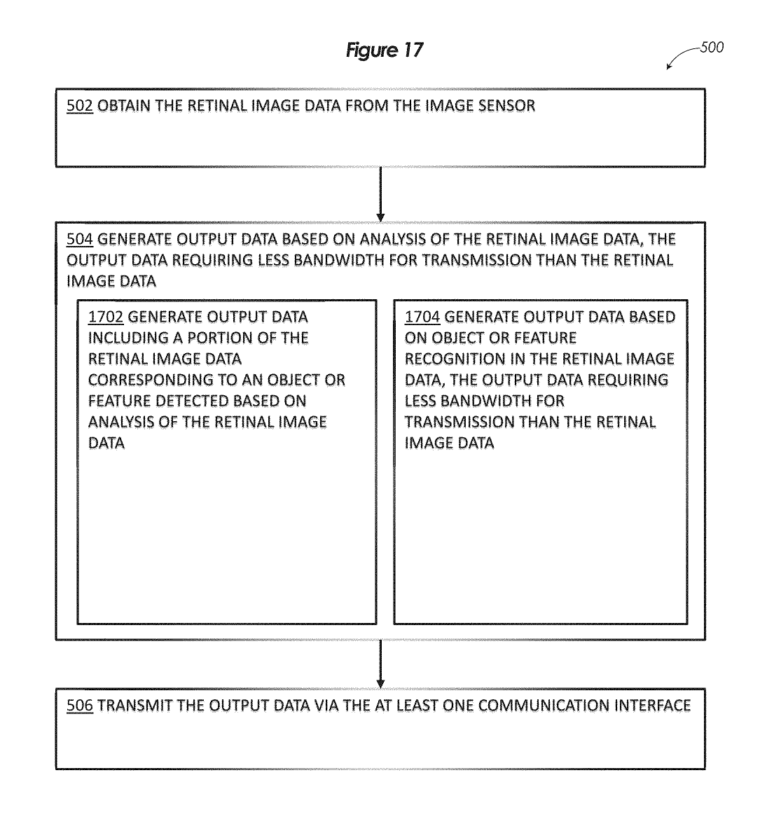

FIG. 17 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data including a portion of the retinal image data corresponding to an object or feature detected based on analysis of the retinal image data at 1702 or generate output data based on object or feature recognition in the retinal image data, the output data requiring less bandwidth for transmission than the retinal image data at 1704.

In one embodiment, the image processor 412 generates output data including a portion of the retinal image data corresponding to an object or feature detected based on analysis of the retinal image data at 1702. The image processor 412 obtains the ultra-high resolution retinal imagery from the image sensor 408 and performs image recognition or analysis to identify a particular object or feature of interest. The image processor 412 can then decimate all or a portion of the pixels outside the area including the particular object or feature of interest. The area can be defined in various ways, including imagery of only the particular object or feature of interest, a percentage or distance around the particular object or feature of interest, a specified box or circle, or the like. The image processor 412 can further reduce the resolution of the imagery of the area corresponding to the particular object or feature of interest and/or can perform one or more other pixel reduction operations (e.g., static object removal, unchanged area removal, overlapping area removal, etc.).

For instance, the image processor 412 can obtain an ultra-high resolution retinal imagery from the image sensor 408 and perform image analysis to identify one or more plaque deposits possibly indicative of Alzheimer's disease. The image processor can select an area of the retinal imagery including the plaque deposits plus approximately 10% beyond the plaque deposits. The non-selected area of the retinal imagery can be decimated and either stored or discarded while the selected area can undergo a pixel reduction and/or compression prior to transmission via the communication interface 410.

In one embodiment, the image processor 412 generates output data based on object or feature recognition in the retinal image data, the output data requiring less bandwidth for transmission than the retinal image data at 1704. The image processor can obtain the ultra-high resolution retinal imagery from the image sensor 408 and perform image recognition to identify a particular object ore feature. In response to detecting the particular object or feature, the image processor 412 can generate output data which may include the relevant portions of the image data and/or other data. Other data generated by the image processor 412 can include a program or function call, alphanumeric text, binary data, or other similar information or action based data.

For instance, the image processor 412 can obtain ultra-high resolution retinal image data from the image sensor 408 and perform object or feature recognition to identify one or inflammation markers, such as narrower retinal arteriolar diameters or larger retinal venular diameters. Upon identifying the one or more markers, the image processor 412 can generate a program function call to initiate a dispensation of a medication, alert a clinical provider, change a diet or exercise schedule (e.g., increase cardiovascular exercise and minimize cholesterol intake), or trigger additional non-retinal physiological measurements.

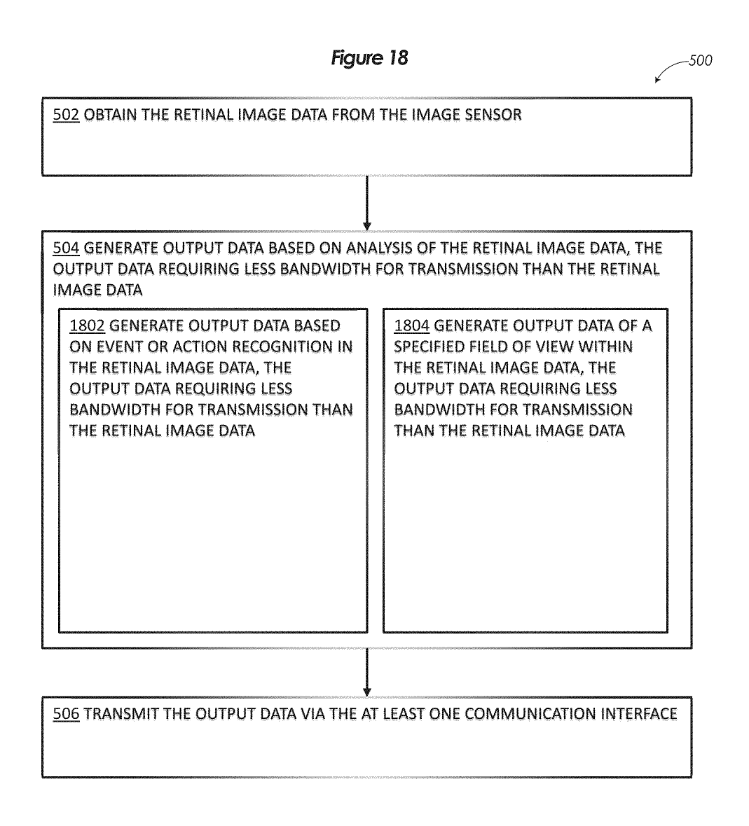

FIG. 18 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data based on event or action recognition in the retinal image data, the output data requiring less bandwidth for transmission than the retinal image data at 1802 or generate output data of a specified field of view within the retinal image data, the output data requiring less bandwidth for transmission than the retinal image data at 1804.

In one embodiment, the image processor 412 generates output data based on event or action recognition in the retinal image data, the output data requiring less bandwidth for transmission than the retinal image data at 1802. The image processor obtains the ultra-high resolution imagery from the image sensor 408 and performs image analysis to identify an event or action, such as a change from a previous retinal image, a measurement beyond a threshold, a deviation from a specified standard, or other defined event or action. Upon detection of the event or action, the image processor 412 generates output data which may include the relevant portions of the image data and/or other data. Other data generated by the image processor 412 can include a program or function call, alphanumeric text, binary data, or other similar information or action.

For instance, the image processor 412 can obtain ultra-high resolution retinal imagery from the image sensor 408 and compare the retinal imagery with one or more previous images obtained at a previous time for the particular subject. In response to the comparison, the image processor 412 can detect vessel discoloration, retinal whitening, and hemorrhages or red lesions not previously present for the subject and possibly indicative of malarial retinopathy. The image processor 412 can then generate a combination of alphanumeric text and binary data based on or in response to the detected change, such as “malarial retinopathy indication: 1”, for transmission via the communication interface 410.

In one embodiment, the image processor 412 generates output data of a specified field of view within the retinal image data, the output data requiring less bandwidth for transmission than the retinal image data at 1804. The image processor 412 obtains the ultra-high resolution retinal imagery from the image sensor 408, but in some cases, not all of the retinal imagery contains useful information. Accordingly, the image processor 412 can perform a reduction operation to eliminate or remove unneeded or non-useful information and retain a field-of-view or selection that contains needed or useful information. Fields of view can include quadrants, sections, segments, radiuses, user defined areas, user requested areas, or areas corresponding to particular features, objects, or events, for example. Fields of view generated by the image processor 412 can also be small, high zoom areas or large, low zoom areas.

For example, the image processor 412 can transmit a large field of view for substantially the entire retinas of both eyes via the communication interface 410 to a client device. A user at the client device can draw a box or pinch and zoom to a specified area of the retina within the large field of view. The client device can present the relatively low resolution specified area of the retina using data previously obtained and further request additional pixel data for the specified area. The image processor 412 can transmit, in response to the client request, additional pixel data, that may have previously been decimated, via the communication interface 410 to enhance the acuity and/or resolution of the specified area at the client device.

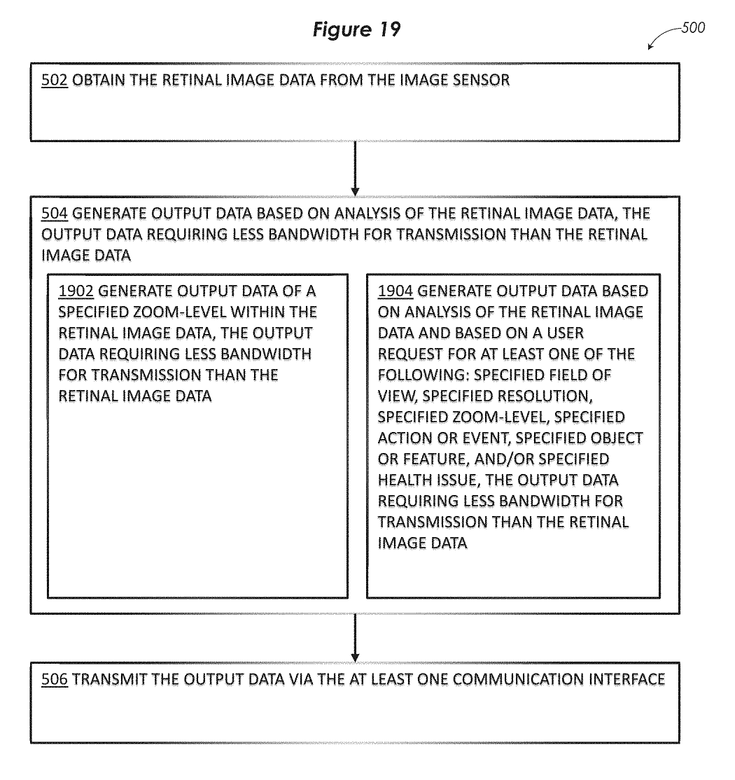

FIG. 19 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data of a specified zoom-level within the retinal image data, the output data requiring less bandwidth for transmission than the retinal image data at 1902 or generate output data based on analysis of the retinal image data and based on a user request for at least one of the following: specified field of view, specified resolution, specified zoom-level, specified action or event, specified object or feature, and/or specified health issue, the output data requiring less bandwidth for transmission than the retinal image data or 1904.

In one embodiment, the image processor 412 generates output data of a specified zoom-level within the retinal image data, the output data requiring less bandwidth for transmission than the retinal image data at 1902. The image processor 412 obtains ultra-high resolution imagery from the image sensor 408 and can digitally generate a specified zoom level by varying the area of retention and varying the pixel retention amount within the retained area. The image processor 412 can enable high zoom levels by retaining more to all of the pixels obtained in the raw retinal image data for a smaller area. The image processor 412 can enable low zoom levels by retaining fewer of the pixels obtained in the raw retinal image data for a larger area. Zoom levels can alternatively be obtained based on mechanical lens adjustment of the optical lens arrangement 404.

For example, the image processor 412 can digitally generate a high-zoom of the optic nerve area of the retina by obtaining the ultra-high resolution retinal imagery, decimating all pixels outside the optic nerve area of the retinal imagery, and retaining most to all of the pixels within the optic nerve area of the retinal imagery. Alternatively, for example, the image processor 412 can digitally generate a low-zoom of the entire retina by obtaining the ultra-high resolution retinal imagery and decimating a portion of the pixels uniformly across the entire retina of the retinal imagery (e.g., every other pixel is removed or a pattern of pixels is removed).

In one embodiment, the image processor 412 generates output data based on analysis of the retinal image data and based on a user request for at least one of the following: specified field of view, specified resolution, specified zoom-level, specified action or event, specified object or feature, and/or specified health issue, the output data requiring less bandwidth for transmission than the retinal image data or 1904. The image processor 412 can be configured to generate output data based on one or more user requests, which one or more user requests can be received via the communication interface 410. The one or more user requests can be a specific request to be satisfied in real-time or near real-time (e.g., a request for a particular field of view and/or zoom level of a retina) or can be a request to be satisfied at a future time (e.g., a request for output data when an action or event occurs, when a feature or object is detected, or pertaining to a particular health issue). Thus, the image processor 412 can be serve response data to user request or can be programmed to perform operations routinely, periodically, in accordance with a schedule, or at one or more specified times in the future. In the instance where the image processor 412 is programmed, the image processor 412 can perform the analysis without further involvement of a user until such time as needed or required.

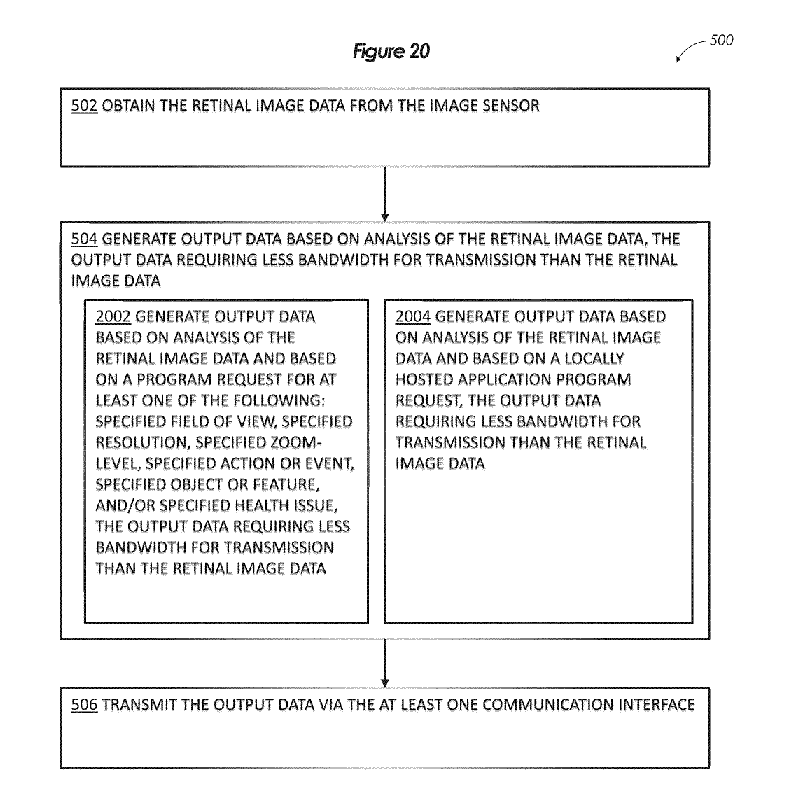

FIG. 20 is a block diagram of a process 500 implemented using a retinal imager device 400 with edge processing, in accordance with various embodiments. In one embodiment, the generate output data based on analysis of the retinal image data, the output data requiring less bandwidth for transmission than the retinal image data at 504 includes one or more of generate output data based on analysis of the retinal image data and based on a program request for at least one of the following: specified field of view, specified resolution, specified zoom-level, specified action or event, specified object or feature, and/or specified health issue, the output data requiring less bandwidth for transmission than the retinal image data at 2002 or generate output data based on analysis of the retinal image data and based on a locally hosted application program request, the output data requiring less bandwidth for transmission than the retinal image data at 2004.

In one embodiment, the image processor 412 generates output data based on analysis of the retinal image data and based on a program request for at least one of the following: specified field of view, specified resolution, specified zoom-level, specified action or event, specified object or feature, and/or specified health issue, the output data requiring less bandwidth for transmission than the retinal image data at 2002. The image processor 412 can receive one or more program requests from a remotely hosted or running application via the communication interface 410. The program request can specify a particular parameter that is executable by the image processor 412 against obtained raw high-resolution retinal imagery data to generate output data. The output data is then transmittable by the image processor 412 to the remote application or to another location (e.g., client or server device).

For example, the image processor 412 can obtain a program request from a third party electronic medical record software application. The program request can include a request for retinal image data of a large field of view and retinal image data of smaller fields of view with a higher zoom level for any detected potential pathology, such as retinal imagery of the optic disk, optic cup, and neuroretinal rim in an event of an abnormal or changing cup-to-disk ratio and share of the neuroretinal rim. The image processor 412 can retain the program request in memory and apply it to obtained retinal image data for a particular patient. In an event of detection of the potential pathology, the image processor 412 can transmit the requested retinal imagery via the communication interface 410 for storage in the electronic medical record software application for the particular patient.

In one embodiment, the image processor 412 generates output data based on analysis of the retinal image data and based on a locally hosted application program request, the output data requiring less bandwidth for transmission than the retinal image data at 2004. The image processor 412 and the computer memory 406 are configurable to host applications, such as third-party applications, that perform one or more specified functions to generate specified output data. Various individuals or entities can create the applications for specialized purposes or research and upload the applications to the fundoscope 402 via the communication interface. The image processor 412 can execute the hosted application alone or in parallel with a plurality of different hosted applications to perform custom analysis and data generation of the ultra-high resolution retinal imagery obtained from the image sensor 408.

For example, a research institution can develop an application that collects non-personal data on the type of retinal pathologies detected versus the duration in outer space. This application can be uploaded to the fundoscope 402 prior to departure of astronauts from Earth. During use of the fundoscope 402 in outer space, the image processor 412 can execute the application during the normal course of retinal image data collection and document detected pathologies and times of the detected pathologies. The output data can be transmitted back to Earth for the research institution via the communication interface 410 without any patient-identifying information. In this example, the same fundoscope 402 can be performing one or more of the operations disclosed herein with respect to a specific astronaut for health monitoring by a clinician. For instance, the image processor 412 can analyze the full resolution retinal imagery and detect an instance of papilledema in the astronaut. Pertinent retinal imagery related to the papilledema can be obtained, reduced, and/or compressed before being transmitted via the communication interface 410 for the clinician.

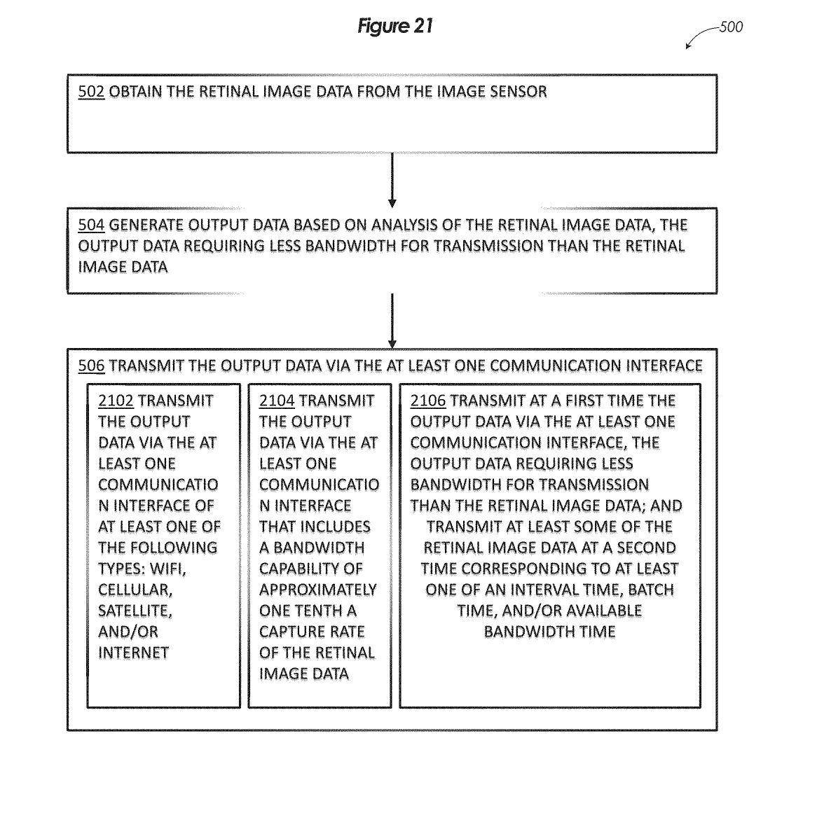

FIG. 21 is a block diagram of a process 500 implemented using a retinal imager device 400 with edge processing, in accordance with various embodiments. In one embodiment, the transmit the output data via the at least one communication interface at 506 includes one or more of transmit the output data via the at least one communication interface of at least one of the following types: WIFI, cellular, satellite, and/or internet at 2102, transmit the output data via the at least one communication interface that includes a bandwidth capability of approximately one tenth a capture rate of the retinal image data at 2104, or transmit at a first time the output data via the at least one communication interface, the output data requiring less bandwidth for transmission than the retinal image data and transmit at least some of the retinal image data at a second time corresponding to at least one of an interval time, batch time, and/or available bandwidth time at 2106.

In one embodiment, the image processor 412 transmits the output data via the at least one communication interface 410 of at least one of the following types: WIFI, cellular, satellite, and/or internet at 2102. The communication interface 410 can be wireless or wired (e.g., ethernet, telephone, coaxial cable, conductor, etc). In instances of wireless communication, the communication interface 410 can include local, ZIGBEE, WIFI, BLUETOOTH, BLE, WIMAX, cellular, GSM, CDMA, HSPA, LTE, AWS, XLTE, VOLTE, satellite, infrared, microwave, broadcast radio, or any other type of electromagnetic or acoustic transmission. The fundoscope 402 can include multiple different types of communication interfaces 410 to accommodate different or simultaneous communications.

In one embodiment, the image processor 412 transmits the output data via the at least one communication interface 410 that includes a bandwidth capability of approximately one tenth a capture rate of the retinal image data at 2104. The image processor 408 can obtain ultra-high resolution imagery from the image sensor 408 at high data rates, such as ten, twenty, thirty, or more gigabytes per second. The communication interface 410 has bandwidth constraints that can be less, significantly less, or orders of magnitude less. For instance, the communication interface 410 can have a bandwidth limitation of approximately one to ten megabytes per second or one gigabyte per second or even as high as five to ten gigabytes per second. In any case, the image processor 412 can have access to more image data than can be timely transmitted via the communication interface 410.

In one embodiment, the image processor 412 transmits at a first time the output data via the at least one communication interface 410, the output data requiring less bandwidth for transmission than the retinal image data and transmits at least some of the retinal image data at a second time corresponding to at least one of an interval time, batch time, and/or available bandwidth time at 2106. The image processor 412 can stagger the transmission of output data via the communication interface 410 or transmit the output data in a single transmission. For instance, the image processor 412 can transmit lower resolution retinal image data, alphanumeric text data, or binary data at a first time to minimize a load on the communication interface 410. Additional pixel data or additional retinal image data can be transmitted by the image processor 412 via the communication interface 410 at a second time. The second time can be scheduled or determined based on one or more parameters, such as available bandwidth above a specified amount or percentage, a user request received, satellite or spacecraft passage over a ground station, level of emergency of a detected pathology, or another similar patient-based, bandwidth-based, or geographic-based parameter.

For example, in a space environment, the fundoscope 402 can be used throughout a space voyage by astronauts to monitor for and detect retinal pathologies. The communication interface 410 may be a WIFI to microwave-based communication channel having a bandwidth constraint of approximately one to ten megabytes per second when the spacecraft passes over an Earth-based ground station. The image processor 412 can obtain retinal image data from the image sensor 408 and perform image analysis to detect one or more potential pathologies. Upon detection, the image processor 412 can immediately transmit via the communication interface 410 an ultra-low bandwidth text-based description of the detected pathology along with astronaut-identifying information. Upon detection of an increased signal strength, such as when positioned over the Earth-based ground station, the image processor 412 can transmit retinal imagery associated with the detected pathology.

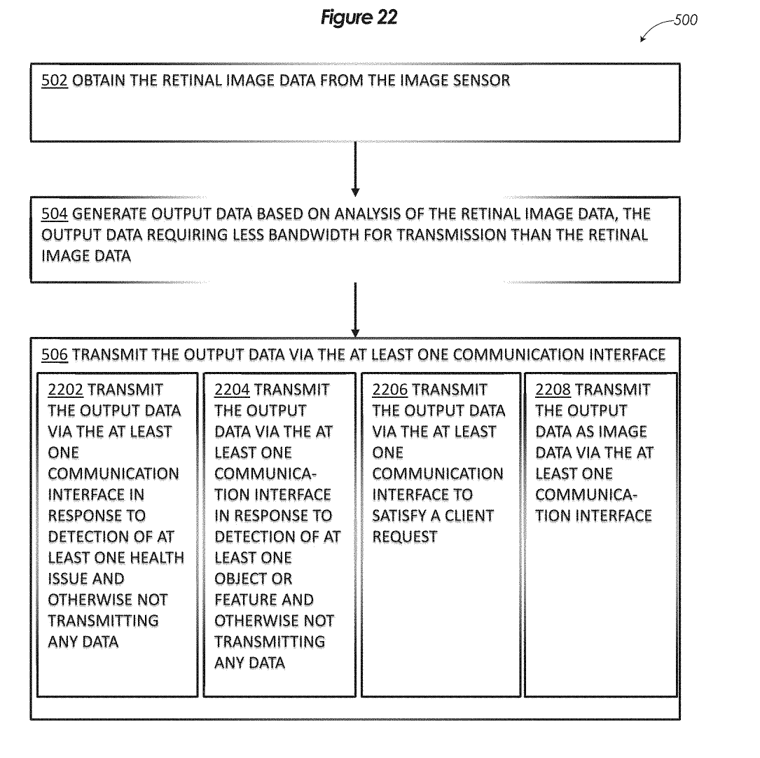

FIG. 22 is a block diagram of a process 500 implemented using a retinal imager device 400 with edge processing, in accordance with various embodiments. In one embodiment, the transmit the output data via the at least one communication interface at 506 includes one or more of transmit the output data via the at least one communication interface in response to detection of at least one health issue and otherwise not transmitting any data at 2202, transmit the output data via the at least one communication interface in response to detection of at least one object or feature and otherwise not transmitting any data at 2204, transmit the output data via the at least one communication interface to satisfy a client request at 2206, or transmit the output data as image data via the at least one communication interface at 2208.

In one embodiment, the image processor 412 transmits the output data via the at least one communication interface 410 in response to detection of at least one health issue and otherwise not transmitting any data at 2202 or transmits the output data via the at least one communication interface 412 in response to detection of at least one object or feature and otherwise not transmitting any data at 2204. The image processor 412 can be programmed to tailor transmitted data to a severity or urgency of a detected pathology, feature, or object in the retinal imagery. For instance, the image processor can transmit retinal imagery and a text or email based notification based on a detected instance of a hemorrhaging blood vessel. Alternatively, the image processor 412 can transmit no information, an alphanumeric text indication, or a binary indication in response to analysis of the retinal imagery data indicating no change, pathology, feature, or object of interest. The scaling of data based on severity or urgency of a detected feature, object, or pathology can serve to make efficient use of the available bandwidth of the communication interface 410. In addition to scaling the information, the image processor 412 can similarity scale the timing of any transmission, such that emergency or urgent information is transmitted more timely than non-urgent or non-emergency information. The image processor 412 can use a combination of time and data quantity adjustments based on one or more outcomes of retinal imagery analysis.

In one embodiment, the image processor 412 transmits the output data via the at least one communication interface 410 to satisfy a client request at 2206. The image processor 412 can respond to one or more client requests received via the communication interface 410. The one or more client requests can include one or more of the following types: field of view, zoom-level, resolution, compression, pathologies to monitor, transmission trigger events, panning, or another similar request. The image processor 412 can respond to the request with a handshake, confirmation, or with the requested information in real-time, near-real time, delayed-time, scheduled-time, or periodic time.

In one embodiment, the image processor 412 transmits the output data as image data via the at least one communication interface 410 at 2208. The image processor 412 can be configured to transmit a variety of data forms, including image data. The image data can be transmitted by the image processor 412 in various forms and formats including any one or more of the following: raster, jpeg, jfif, jpeg 2000, exif, tiff, gif, bmp, png, ppm, pgm, pbm, pnm, webp, hdr, heif, bat, bpg, vector, cgm, gerber, svg, 2d vector, 3d vector, compound format, stereo format.

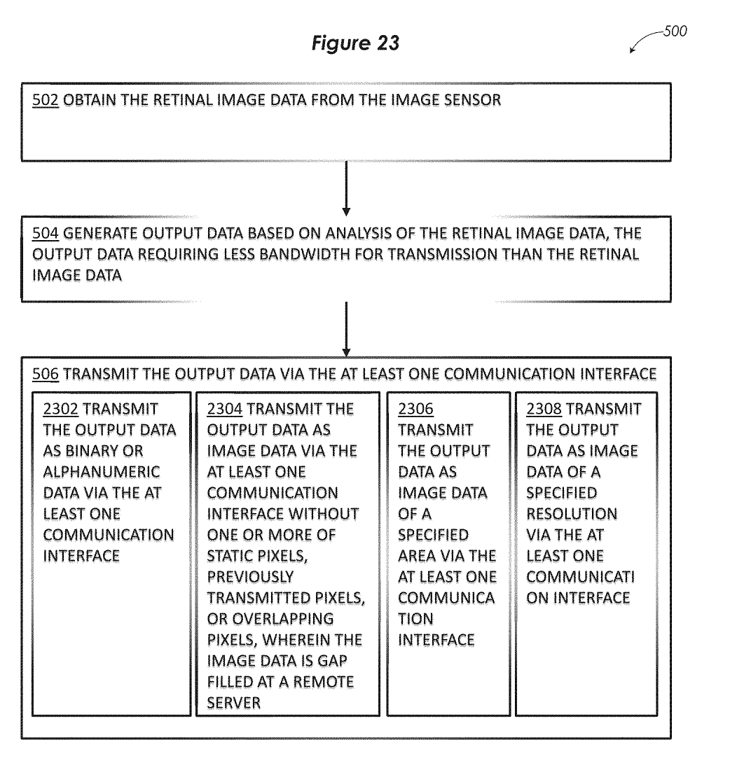

FIG. 23 is a block diagram of a process 500 implemented using a retinal imager device 400 with edge processing, in accordance with various embodiments. In one embodiment, the transmit the output data via the at least one communication interface at 506 includes one or more of transmit the output data as alphanumeric or binary data via the at least one communication interface at 2302, transmit the output data as image data via the at least one communication interface without one or more of static pixels, previously transmitted pixels, or overlapping pixels, wherein the image data is gap filled at a remote server at 2304, transmit the output data as image data of a specified area via the at least one communication interface at 2306, or transmit the output data as image data of a specified resolution via the at least one communication interface at 2308.

In one embodiment, the image processor 412 transmits the output data as alphanumeric or binary data via the at least one communication interface 410 at 2302. The image processor 412 can transmit binary or alphanumeric output data derived from or based on the retinal image data instead of or in addition to transmitting the retinal image data. The alphanumeric text can include words, phrases, paragraphs, artificial intelligence-generated statements, sentences, symbols, numbers, or the like. Binary data can include any of the following: on, off, high, low, 0, 1, yes, no, or other similar representations of binary values.

In one embodiment, the image processor 412 transmits the output data as image data via the at least one communication interface 410 without one or more of static pixels, previously transmitted pixels, or overlapping pixels, wherein the image data is gap filled at a remote server at 2304. The image processor 412 can transmit retinal image data that is then retained or stored at a remote location, such as a network location, server, or client device. The transmission by the image processor 412 can be in response to a client request, a program request, a scheduled transmission or can be accomplished during low bandwidth or low activity periods. Following transmission of the retinal image data, the image processor 412 can obtain new retinal image data from the image sensor 408 and perform analysis to determine when any of the retinal image data has previously been transmitted. The image processor 412 can remove any identified previously transmitted retinal image data and retain only changed or non-previously transmitted retinal image data. The image processor 412 can then transmit the changed or non-previously transmitted retinal image data via the communication interface 410, such that the previously transmitted retinal image data is gap-filled, combined, or inserted to establish a composite retinal image prior to display or print output.

For example, the image processor 412 can obtain retinal image data from the image sensor 408 for John Q. Smith. The retinal image data includes no pathological indications or unusual biomarkers, deposits, or discolorations. A server device receives the retinal image data for John Q. Smith and stores it in memory. During a subsequent fundoscope session, the image processor 412 obtains retinal image data from the image sensor 408 for John Q. Smith. During this session, the image processor 408 identifies one or more instances of hemorrhaging. Instead of transmitting all of the retinal image data, the image processor 412 decimates all unchanged pixels of the retinal image other than the area surrounding the hemorrhaging. The image processor 412 then transmits the retinal image data corresponding to the hemorrhaging and the server gap-fills the previously transmitted retinal image data to recreate the composite retinal image data for John Q. Smith.

In one embodiment, the image processor 412 transmits the output data as image data of a specified area via the at least one communication interface 410 at 2306. The image processor 412 can determine the specified area from a client request, a program request, or can be determined in response to a detected pathology. Client requests for areas can be received via the communication interface 410 and include coordinates, vector values, raster image drawings, text, binary, or other data. Program requests can be provided manually or automatically by one or more programs that may be resident on the fundoscope 402 or on a remote computer, server, cloud, or client device. The program requests can similarly include coordinates, vector values, raster image drawings, text, binary, or other data. The program requests can be triggered in response to detected values, pathologies, indications, or measurements.

For example, the image processor 412 can obtain retinal image data and perform image analysis to detect an instance of a choroidal fold. An application program request can be generated automatically to obtain measurements, generate a textual description of the choroidal fold, and retain high-zoom level retinal image data pertaining to the choroidal fold for transmission via the communication interface 410 for a client device output.

In one embodiment, the image processor 412 transmits the output data as image data of a specified resolution via the at least one communication interface 410 at 2308. The image processor 412 can determine specified resolutions from metadata attached to a client request, identification of a client device associated with a client request, a previous specified resolution, an average resolution, or a default resolution. The image processor 412 can apply the specified resolution uniformly or non-uniformly to retinal image data.

For example, a client device can request retinal image data at a 1600×1200 pixels. The image processor 412 can apply the specified resolution to the pixel retention of the retinal image data non-uniformly such that the areas surrounding the optic nerve head, the fovea, the macula, and the venules and arterioles are reduced to 1600×1200 pixels. However, the image processor 412 can further reduce other areas of the retinal image data to less than 1600×1200, such as to 300×200 pixels. The image processor 412 can transmit the non-uniform resolution retinal image data to the client device at a first time and then follow up with full 1600×1200 retinal imagery at a later second time (e.g., immediately thereafter the first time).

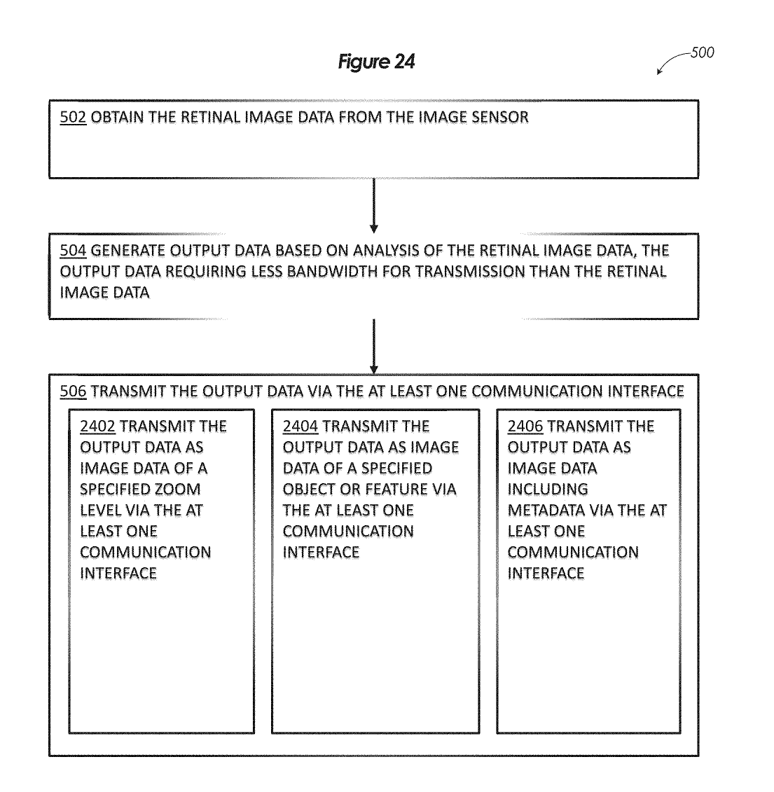

FIG. 24 is a block diagram of a process 500 implemented using a retinal imager device 400 with edge processing, in accordance with various embodiments. In one embodiment, the transmit the output data via the at least one communication interface at 506 includes one or more of transmit the output data as image data of a specified zoom level via the at least one communication interface at 2402, transmit the output data as image data of a specified object or feature via the at least one communication interface at 2404, or transmit the output data as image data including metadata via the at least one communication interface at 2406.

In one embodiment, the image processor 412 transmits the output data as image data of a specified zoom level via the at least one communication interface 410 at 2402. The image processor 412 can obtain a specified zoom level from a client request, program request, or in response to a detected parameter. For instance, the specified zoom level can be a percentage or level (e.g., 10% or 90% zoom, low or high-level zoom). The specified zoom level can include a specified area as well as a specified visual acuity for that particular area. The specified area can be defined by a default area, a selected area, a box, a focus center, an anatomical structure, or a pathological area. The image processor 412 can also generate a specified zoom level in anticipation of a client or program request and transmit at least some of the anticipated zoom level data prior to the client or program request to reduce future latency.

For example, the image processor 412 can respond to a client request and provide retinal image data corresponding to a low-zoom substantially entire field of view of the retina. The image processor 412 can also detect through image analysis an instance of a plaque or discoloration in the retinal image data. The image processor 412 can begin transmitting high-zoom level retinal image data corresponding to the plaque or discoloration prior to any user request in anticipation that a request for the zoom will be forthcoming. If and when a user request for high-zoom retinal image data corresponding to the plaque or discoloration is received, the image processor 412 can already have transmitted some or all of the retinal image data.

In one embodiment, the image processor 412 transmits the output data as image data of a specified object or feature via the at least one communication interface at 2404. The image processor 412 can receive an indication of a specified object or feature from a user request, a program request, or based on a detected pathology or variation in the retinal image data. The specified object or feature can be an anatomical feature, a biomarker, or an area corresponding to a detected pathology, change, or variation. The image processor 412 can select and transmit only the retinal image data associated with the specified object or feature or can transmit additional retinal image data. For instance, the image processor 412 can transmit retinal image data corresponding to an object or feature in addition to retinal image data corresponding to one or more other instances of the object or feature.

For example, the image processor 412 can receive a user request for retinal image data corresponding to a particular engorged arteriole. The image processor 412 can select and transmit the retinal image data corresponding to the particular engorged arteriole, but also select and transmit unrequested portions of the retinal image data. The unrequested portions of the retinal image data can be determined by the image processor 412 to relate to the requested portions, such as retinal image data corresponding to all engorged venules or arterioles. A client device can then receive the transmitted selected retinal image data and the unselected retinal image data related to the selected retinal image data for display.

In one embodiment, the image processor 412 transmits the output data as image data including metadata via the at least one communication interface 410 at 2406. The metadata generated, selected, or identified by the image processor 412 can depend on one or more factors, including client specification, program specification, a particular patient, or detected pathologies, markers, features, or objects associated with the retinal image data. The metadata can include text, numbers, symbols, links, images, or other similar data that describes or relates to the retinal image data. The metadata can also include information regarding time, omitted image data, location of previously transmitted image data, data size, bandwidth requirements, frame rate, resolution, file type, or the like.

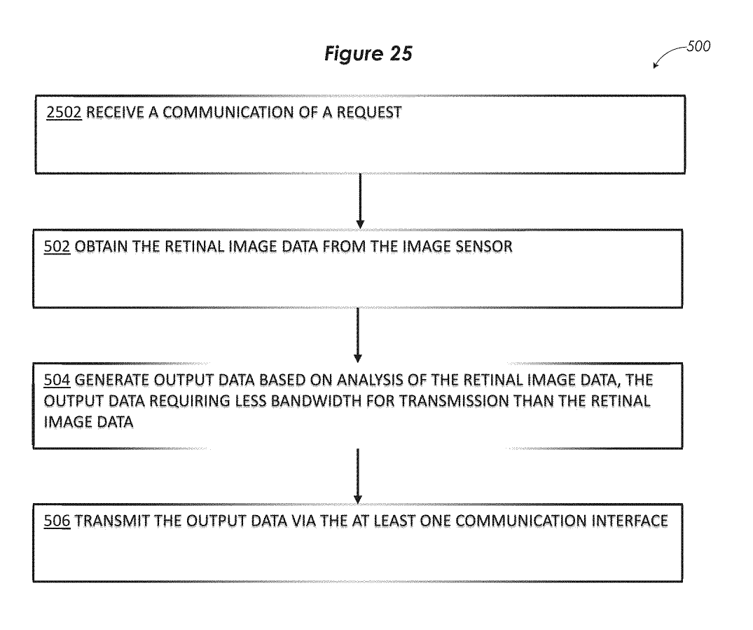

FIG. 25 is a block diagram of a process 500 implemented using a retinal imager device 400 with edge processing, in accordance with various embodiments. In one embodiment, the process 500 further includes an operation of receive a communication of a request at 2502.

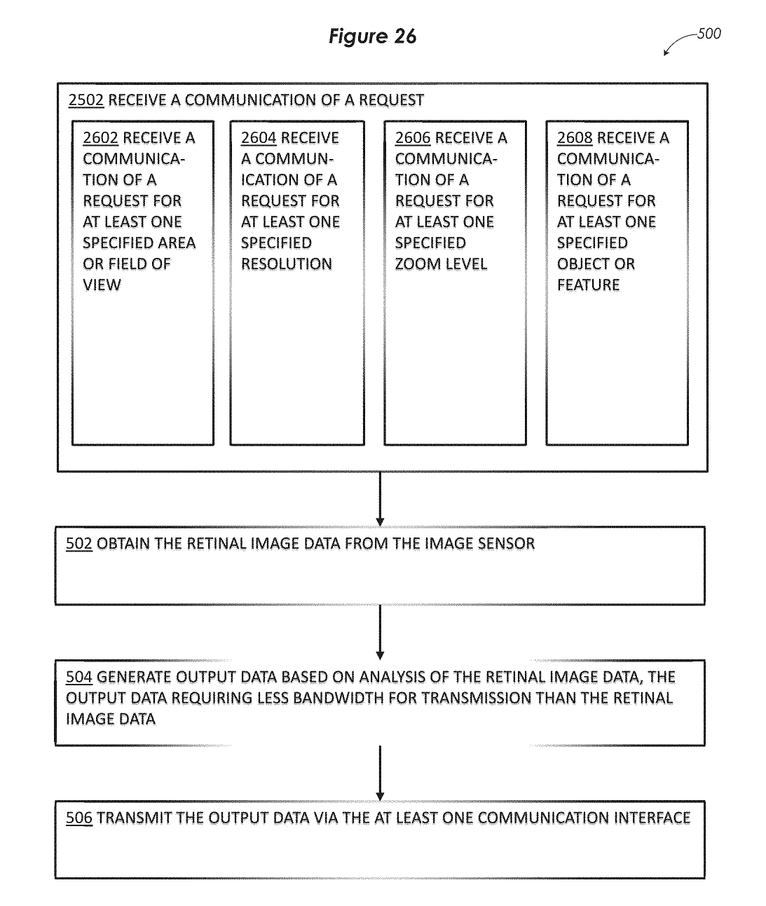

FIGS. 26-28 are block diagrams of a process 500 implemented using a retinal imager device 400 with edge processing, in accordance with various embodiments. In one embodiment, the receive a communication of a request at 2502 includes one or more of receive a communication of a request for at least one specified area or field of view at 2602, receive a communication of a request for at least one specified resolution at 2604, receive a communication of a request for at least one specified zoom level at 2606, receive a communication of a request for at least one specified object or feature at 2608, receive a communication of a request involving zooming at 2702, receive a communication of a request involving panning at 2704, receive a communication of a request for at least one specified action or event at 2706, receive a communication of a program request at 2708, or receive via the at least one communication interface a communication of a client request at 2802.

The image processor 412 can receive via the at least one communication interface 410 a communication of a client request at 2802. The client request can be received directly or indirectly via a communication network from a client device. Client devices can include any one or more of a smartwatch, a smartphone, a mobile phone, a tablet device, a laptop device, a computer, a server, an augmented reality headset, a virtual reality headset, a game console, or a combination of the foregoing. The communication network can include a direct wire link, a direct wireless link, an indirect wire link, an indirect wireless link, the Internet, a local network, a wide area network, a virtual network, a cellular network, a satellite network, or a combination of the foregoing.

In the context of a client device, the image processor 412 can receive from the client device a request for at least one specified area or field of view at 2602, at least one specified resolution at 2604, at least one specified zoom level at 2606, at least one specified object or feature at 2608, zooming at 2702, panning at 2704, or at least one specified action or event at 2706. The requests can be transmitted in audio, binary, or alphanumeric text form and can be generated from voice input, graphical selection, physical control movement, device movement or tilt, finger gesture, sensor input, or another source.

For example, in one particular embodiment, a client device provides a user interface associated with one or more fundoscopes 402. A particular fundoscope can be selected from the one or more fundoscopes 402 to obtain retinal image data from that particular fundoscope 402. Retinal image data is obtained and displayed from the fundoscope 402 in real-time or near-real-time for a particular individual being analyzed. The retinal imagery data is output for display and can be interacted with through a combination of graphical user interface elements, input fields, gestures, and/or movements of the client device. The graphical user interface elements can include buttons or sliding bars, such as to enable control of zoom, pan, resolution, or other parameters. The input fields can enable text entry, such as a number value for a zoom level or a specific object to anchor the field of view. Gestures and device movement can be combined to enable functions, such as panning by movement of the client device, zooming by pinching opposing fingers on the touch screen, and/or switching between retinas of the particular individual by swiping a finger. Voice input can be accepted to instruct the particular individual with respect to particular actions, such as to communicate with the particular individual and inform that individual to move, shift, change eyes, stay still, or another instruction. The client device can also provide notifications and/or alerts regarding the availability of retinal image data or regarding potential detected pathologies, changes, or variations associated with retinal image data.

The image processor 412 can receive a communication of a program request at 2708. The program can be running on the fundoscope 402 and/or running on a client device, computer, server, or in a cloud environment. In embodiments, where the program is running on a remote client device, computer, server, or in a cloud environment, the program request can be received directly or indirectly via a communication network. The communication network can include a direct wire link, a direct wireless link, an indirect wire link, an indirect wireless link, the Internet, a local network, a wide area network, a virtual network, a cellular network, a satellite network, or a combination of the foregoing. The program can be a special-purpose program dedicated to obtaining, storing, analyzing, forwarding, or otherwise processing retinal image data for one or more individuals. Alternatively, the program can be part of another general purpose or specialized purpose application or system, such as an electronic medical records system, a health and physiology monitoring program, a home health system, or the like.

For example, the fundoscope 402 can host a plurality of third party applications that each perform different analyses and operations with respect to retinal image data obtained from the image sensor 408. Potential third party applications can include research applications, commercial applications, pharmaceutical applications, consumer or hobby applications, or other scientific applications. Each of the applications can obtain some or all of the retinal image data and independently perform different operations thereon. For instance, one application may request, store, transmit, and/or analyze retinal image data of a particular field of view (e.g., optic disk area only for researchers in the field of diet-induced changes to the optic disk controlled for age). Another certain application may request, store, transmit, and/or analyze retinal image data pertaining only to certain features (e.g., retinal imagery of plaques when present for control and non-control groups of individuals taking part in a study involving a particular Alzheimer's disease drug). Another application may request, store, transmit and/or analyze retinal image data of medium resolutions for all individuals without any person-identifying information (e.g., a medical school may want real-time imagery to present during an ophthalmology lecture during class). Thus, a variety of customized specific third-party applications can be developed and hosted on the fundoscope 402 for a variety of different entities to perform specific functions and generate different outputs based on the same retinal imagery data.

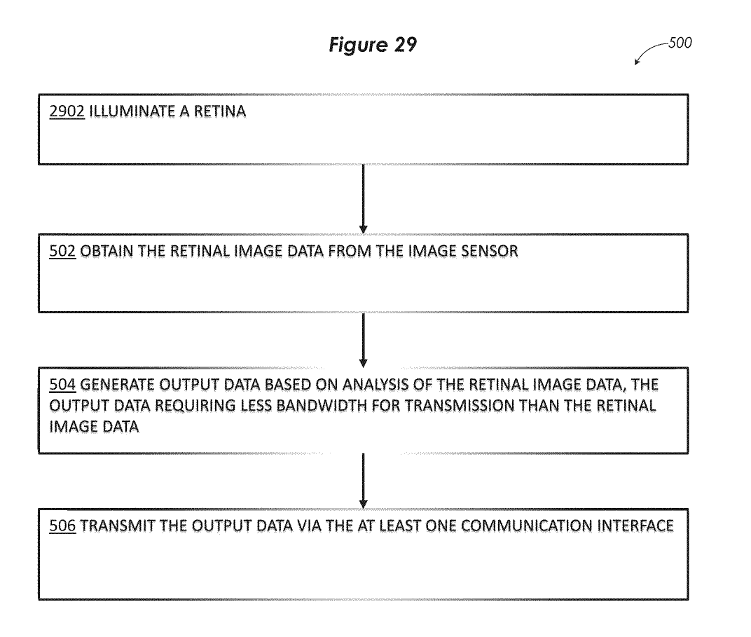

FIG. 29 is a block diagram of a process 500 implemented using a retinal imager device 400 with edge processing, in accordance with various embodiments. In one embodiment, the process 500 further includes an operation of illuminate a retina at 2902. The optical lens arrangement 404 can include an illumination source, such as an incandescent light, an organic light emitting diode, a light emitting diode, a laser, or another light source or combination of light sources.

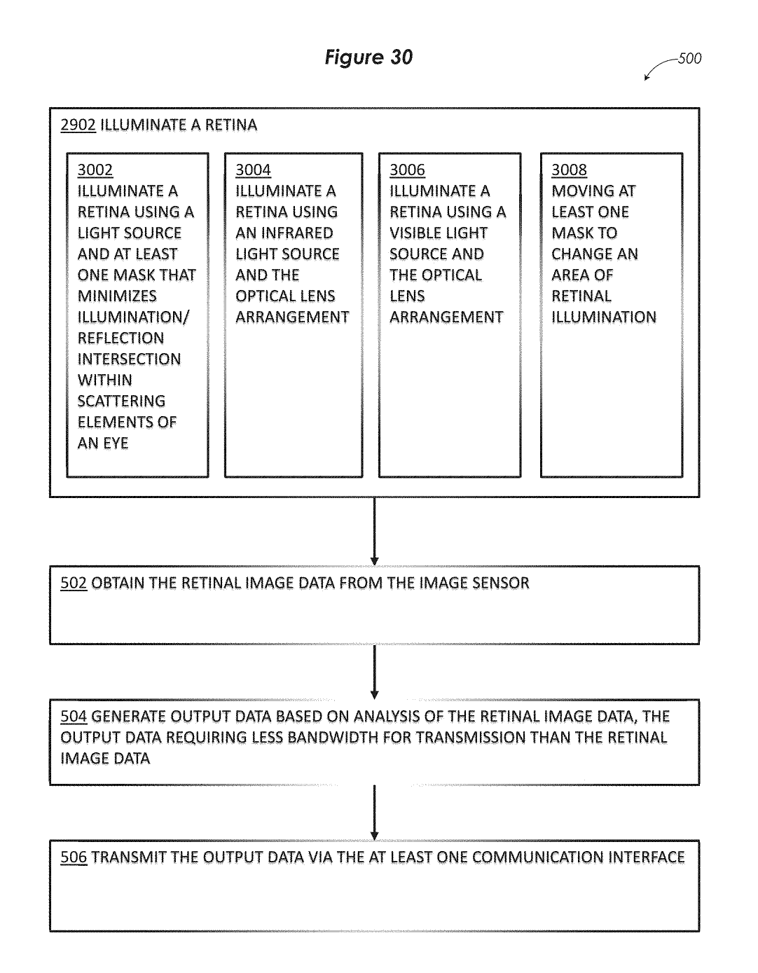

FIG. 30 is a block diagram of a process 500 implemented using a retinal imager device 400 with edge processing, in accordance with various embodiments. In one embodiment, the illuminate a retina at 2902 includes one or more of illuminate a retina using a light source and at least one mask that minimizes illumination/reflection intersection within scattering elements of an eye at 3002, illuminate a retina using an infrared light source and the optical lens arrangement at 3004, illuminate a retina using a visible light source and the optical lens arrangement at 3006, or moving at least one mask to change an area of retinal illumination at 3008.

In one embodiment, the optical lens arrangement 404 illuminates a retina using a light source and at least one mask that minimizes illumination/reflection intersection within scattering elements of an eye at 3002. The optical lens arrangement includes a light source that is directed onto the retina and reflected for imaging. The intersection of the illumination light and the reflected light is minimized in the cornea and lens structures of the eye through use of one or more masks that block at least some of the illumination light. The masks can be constructed from any light obstructing material and may be partially or fully obstructive to light.

In one embodiment, the optical lens arrangement 404 illuminates a retina using an infrared light source at 3004. The infrared light source can include an infrared light emitting diode, an infrared organic light emitting diode, a laser, or another infrared light source. The infrared light is directed onto the retina via the optical lens arrangement and reflected for infrared imaging. Infrared light does not trigger the same iris constriction response and can therefore be used prior to visible imaging for eye positioning or repositioning, focus, or other operation where iris constriction is to be avoided or limited. The infrared light source can include one or more masks that at least partially obscure the infrared light to minimize the intersection of the illumination infrared light and reflected infrared light within the scattering elements of the eye (e.g., cornea and lens).

In one embodiment, the optical lens arrangement 404 illuminates a retina using a visible light source at 3006. The visible light source can include a light emitting diode, an organic light emitting diode, an incandescent light, a laser, or another visible light source. In certain embodiments, the visible light source is limited to a certain wavelength (e.g. white or red). The visible light source is directed via the optical lens arrangement 404 as illumination light onto the retina where it is reflected for retinal imaging. One or more masks are used to at least partially obscure the visible light to limit the intersection of the illumination light and the reflected light within the scattering elements of the eye (e.g. cornea and lens). Minimization can be less than a certain percentage, for example less than 1% or less than 5% or less than 10% or less than 25% interaction between the illumination light and the reflected light within the scattering elements of the eye. In certain embodiments, the visible light source is emitted for retinal imaging following focus and/or eye positioning performed using an infrared light source.

In one embodiment, the optical lens arrangement 404 moves at least one mask to change an area of retinal illumination at 3008. The use of at least one mask can limit the illumination on certain parts of the retina. In certain embodiments, the at least one mask is moved over the course of retinal imaging (e.g., smoothly or stepped over video retinal imagery capture or to different prespecified locations between static imagery capture). The captured retinal imagery over time or from different images can then be used to create a complete composite retinal image by retaining the portions with high acuity and stitching those retained portions together, for example.

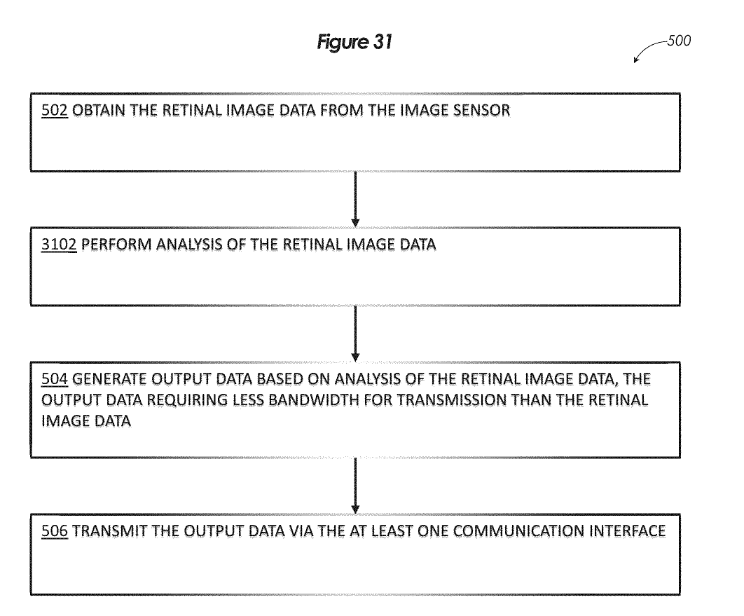

FIG. 31 is a block diagram of a process 500 implemented using a retinal imager device 400 with edge processing, in accordance with various embodiments. In one embodiment, the process 500 further includes an operation of perform analysis of the retinal image data at 3102. The image processor 412 can perform the analysis of the retinal image data in the course of performance of one or more operations illustrated or disclosed herein. The analysis can include one or more of image recognition, image comparison, feature extraction, object recognition, image segmentation, motion detection, image preprocessing, image enhancement, image classification, contrast stretching, noise filtering, histogram modification, or other similar operation.

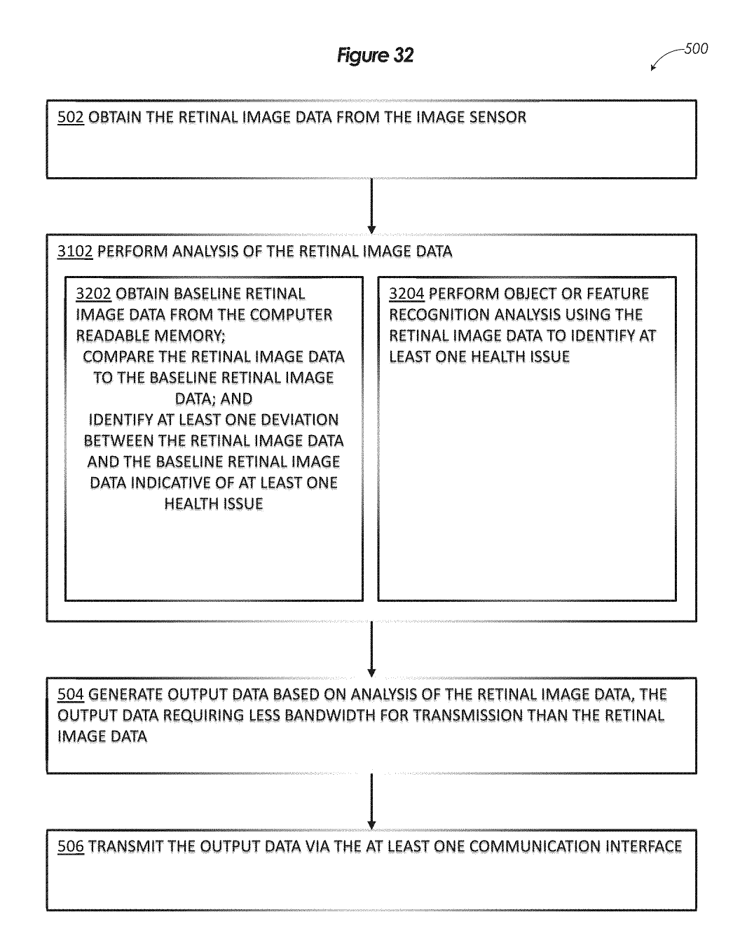

FIG. 32 is a block diagram of a process 500 implemented using a retinal imager device 400 with edge processing, in accordance with various embodiments. In one embodiment, the perform analysis of the retinal image data at includes one or more of obtain baseline retinal image data from the computer readable memory, compare the retinal image data to the baseline retinal image data, and identify at least one deviation between the retinal image data and the baseline retinal image data indicative of at least one health issue at 3202 or perform object or feature recognition analysis using the retinal image data to identify at least one health issue at 3204.

In one embodiment, the image processor 412 obtains baseline retinal image data from the computer readable memory 406, compares the retinal image data to the baseline retinal image data, and identifies at least one deviation between the retinal image data and the baseline retinal image data indicative of at least one health issue at 3202. The image processor 412 can obtain retinal image data at a first time for a particular individual and store that retinal image data in the computer memory 406 as the baseline retinal image data. At a second time after the first time, the image processor 412 can obtain new retinal image data and compare the retinal image data to the baseline retinal image data of the first time stored in the computer memory 406. The image processor 412 can identify a change or deviation between the retinal image data and the baseline retinal image data, which may be indicative of a health issue. Health issues have been illustrated and discussed herein and can include, for example, one or more of diabetic retinopathy, macular degeneration, cardiovascular disease, glaucoma, malarial retinopathy, Alzheimer's disease, globe flattening, papilledema, and/or choroidal folds. Upon detection or non-detection of a health issue, the image processor 412 can perform one or more of the operations illustrated and/or disclosed herein. In certain embodiments, the baseline retinal image data can be for a different individual or associated with a normal retina.

In one embodiment, the image processor 412 performs object or feature recognition analysis using the retinal image data to identify at least one health issue at 3204. The image processor 412 can perform object or feature recognition analysis with or without a corresponding image baseline comparison analysis. The object or feature recognition can include identifying anatomical structures, biomarkers, discolorations, measurements, shapes, contours, lines, or the like within any of the retinal image data. The objects or features can be associated with various potential health issues and used by the image processor 412 to identify a potential health issue or array of possible potential health issues. Again, potential health issues have been disclosed and illustrated herein, but can include diabetic retinopathy, macular degeneration, cardiovascular disease, glaucoma, malarial retinopathy, Alzheimer's disease, globe flattening, papilledema, and/or choroidal folds. Upon detection of the potential health issue, the image processor 412 can perform one or more operations as discussed and/or illustrated herein.

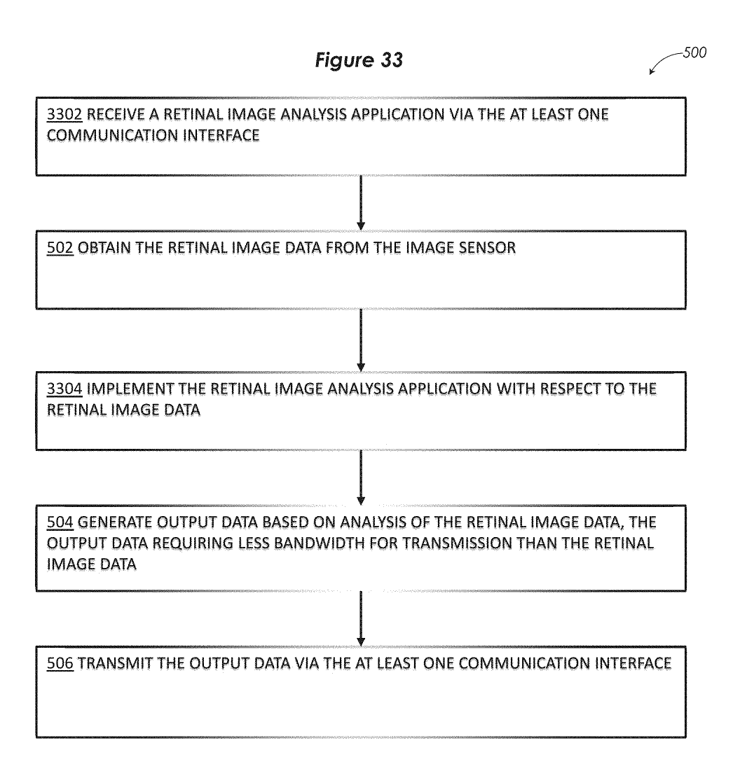

FIG. 33 is a block diagram of a process 500 implemented using a retinal imager device 400 with edge processing, in accordance with various embodiments. In one embodiment, the process 500 further includes operations of receive a retinal image analysis application via the at least one communication interface at 3302 and implement the retinal image analysis application with respect to the retinal image data at 3304. The image processor 412 of the fundoscope 402 is not necessarily static in its configuration. Instead, the image processor 412 can be programmed to perform special purpose operations that change over time by receiving software applications via the communication interface 410 and deploying the software applications for specialized analysis and output of the retinal image data. The customization of the image processor 412 configuration enables modifications over time to any of the amount and timing of retinal image data collection, mask movement, illumination intensity or duration or wavelength, pixel decimation, pixel selection, object removal, unselected retinal imagery transmission, anticipated object or area transmissions, gap-filling, image analysis, data generation, data output, output data destination or timing, bandwidth usage, feature or object detection, event triggers, comparison or health issue detection algorithms, health issue focuses, retinal areas of interest, or the like. Entities such as companies, individuals, research institutions, scientific bodies, consumer groups, educational institutions, or the like can therefore develop specialized applications based on their respective needs and upload the specialized applications to the fundoscope 402 for implementation in parallel or series via the image processor 412. The applications can be updated, deleted, stopped, started, or otherwise controlled as needs change over time.

For example, a pharmaceutical company interested in understanding cardiovascular disease in a population of individuals ages 40-50 can develop an application that collects summary alphanumeric text data regarding age of patient and type of retinal markers indicative of cardiovascular disease detected. This application can be uploaded to the fundoscope 402 or an array of fundoscopes 402 used in a cardiology clinics and hospital wards. During use of the fundoscope 402, the image processor 412 can execute the application during the normal course of retinal image data collection and document the requested data. The output data can be transmitted back to a computer destination for the pharmaceutical company to be used for research or commercialization decisions. In this example, the same fundoscope 402 can be performing one or more of the operations disclosed herein with respect to a specific patient for real-time or near-real time health analysis or monitoring by a clinician and can be executing one or more other third-party applications for one or more different entities with different data outputs.

In one particular embodiment, as an additional example of unobtrusive monitoring of retinal regions for medical diagnostic functions, the retinal imager 402 can be used in coordination with with fluorescence to identify particular indications. For example, fluorescent tagged proteins or fluorescent chemicals can be introduced into the eye globe via the sclera and vitreous humor (e.g., via an eye drop or needle). Alternatively, the fluorescent tagged proteins or fluorescent chemicals can be introduced via blood flow to the retina (e.g., capsule, pill, consumable, or IV injection). The fluorescent chemical or protein adheres to certain pathological indications of the retina and can be captured via illumination and imaging via the image sensor 408. The image processor 412 determines and detects the presence of the fluorescent tagged proteins or fluorescent chemicals and can generate output data as discussed and illustrated herein based on the same. As one particular example, curcumin has been shown to adhere to amyloid plaques and will fluoresce in response to the proper optical stimulation. Thus optical stimulation of the retina or other near surface blood flows in conjunction with curcumin fluoresce can be an indicator of potential Alzheimer's disease. In the event of detection of curcumin fluoresce, the image processor 412 can generate output data, such as high visual acuity retinal imagery of areas of the retina associated with the detected curcumin or such as a binary indication of potential Alzheimer's disease.

In other embodiments, the retinal imager or fundoscope 402 can be used to perform unobtrusive medical diagnostic functions through non-retinal eye or facial monitoring. For instance, the imager 402 can be positioned with, attached to, incorporated in, or integrated into a vehicle, such as a car, truck, airplane, boat, train, heavy machinery, etc, with a field of view directed at a driver, passenger, or occupant of the vehicle. The imager 402 can then monitor and/or detect eye movements, pupil size, dilation, blinking, eye lid position or movement, facial expression, facial features, skin coloration, or other eye, head, neck, or face parameter. This information, optionally in combination with other driver awareness sensors, can be used to perform diagnostic functions, such as determine driver awareness, alertness, drowsiness, sickness, drug use, alcohol use, energy, or heath. Based on the outcome of any diagnostic function, the imager 402 can inform the activation of stimulation routines, such as via digital games, displays, body worn stimulators, audio devices, an illumination source, or the like. The imager 402 can monitor and/or detect responses to stimulation and make adjustments to the stimulation or initiate control of other devices or equipment based on the same. For example, the imager 402 can monitor dilation or pupil size of a driver's eyes. In response to a determination that the dilation response is slow, fluctuating, unstable, abnormal, or above or below a specified threshold level, the imager 402 can signal an LED repeatedly or periodically while monitoring the dilation response. The imager 402 can obtain measurements of the dilation or pupil size of the driver's eyes from before, during, and after stimulation, and determine from this information, and optionally from other sensor inputs, whether the driver is suffering from or experiencing fatigue, whether the driver may have another health issue, or whether the driver is intoxicated or under the influence of drugs. Based on a determination of fatigue, the imager 402 can signal a music player, roll a window down, adjust a seat position, slow the vehicle, set a limit on vehicle use (e.g., shut down after 30 miles), notify a 3^{rd}-party, record the data, initiate a phone call, or other similar action to mitigate or address the fatigue.

In one particular embodiment, the imager 402 is configured to perform eulerian video magnification in the context of retinal imagery, facial imagery, or body part imagery. The imager 402 captures one or more images or videos of the individual and magnifies one or more of color changes or movement within the one or more images or videos. For instance, the imager 402 can generate a video of the retina or face where the pulse, pulse strength, or pulse duration is detectable and/or measurable through magnification of the color changes. As another example, the imager 402 can generate a video of a neck or arm of an individual where pulse, pulse strength, or pulse duration is detectable and/or measurable through magnification of skin perturbances. The image sensor 402 can use pulse, pulse rate, pulse strength, or other information obtained through the eulerian video magnification to identify instances of stress, anxiety, fatigue, attentiveness, illness, sickness, disease, or other health issue. The imager 402 can signal or control one or more devices based on any identified or detected parameter or health issue, including signaling an alert, signaling for an additional parameter measurement, capturing imagery, generating imagery, transmitting imagery, controlling a medication dispenser, controlling a climate control device, controlling a vehicle, or the like. In one particular example, the imager 402 obtains retinal image data as video data from the image sensor 408. The image processor 412 performs eulerian video magnification of the retinal imagery obtained to accentuate, exaggerate, or magnify the blood flow within the retina. The image processor 412 then performs image analysis on the retinal image data to determine pulse rate, strength, and any changes in blood flow from one or more prior images. The image processor 412 can generate output data based on a pulse rate or strength that is above or below a specified threshold or a detected change over time in the blood flow, which output data can include any of that discussed or illustrated herein. Such output data can include, for example, a notification to a clinician of the abnormal pulse rate or strength or retinal imagery surrounding a potential hemorrhage site.

The present disclosure may have additional embodiments, may be practiced without one or more of the details described for any particular described embodiment, or may have any detail described for one particular embodiment practiced with any other detail described for another embodiment. Furthermore, while certain embodiments have been illustrated and described, as noted above, many changes can be made without departing from the spirit and scope of the disclosure.

1. A machine-vision enabled fundoscope for retinal analysis, the fundoscope comprising:
an optical lens arrangement;
an image sensor positioned with the optical lens arrangement and configured to convert detected light to retinal image data;
computer readable memory;
at least one communication interface; and
an image processor communicably linked to the image sensor, the computer readable memory, and the at least one communication interface, the image processor programmed to execute operations including at least:
obtain the retinal image data from the image sensor;
generate output data based on analysis of the retinal image data, the output data requiring less bandwidth for transmission than the retinal image data; and
transmit the output data via the at least one communication interface.

2-7. (canceled)

8. The fundoscope of claim 1, wherein the optical lens arrangement comprises:
an imaging optical lens arrangement aligned in a first axis;
an illumination lens arrangement aligned in a second axis that is perpendicular to the first axis; and
at least one polarizing splitter/combiner.

9. The fundoscope of claim 8, further comprising:
an illumination LED configured to emit light; and
one or more masks configured to obscure at least some of the light of the illumination LED prior to passing through the illumination lens arrangement to minimize illumination/reflection intersection within scattering elements of an eye,
wherein the at least one polarizing splitter/combiner is configured to redirect the light passing through the illumination lens arrangement aligned in the second axis into the imaging optical lens arrangement aligned in the first axis to illuminate at least one portion of the retina.

10. The fundoscope of claim 9, further comprising:
an infrared LED configured to emit infrared light; and
one or more infrared masks configured to obscure at least some of the infrared light of the infrared LED prior to passing through the illumination lens arrangement to minimize illumination/reflection intersection within scattering elements of an eye,
wherein the at least one polarizing splitter/combiner is configured to redirect the infrared light passing through the illumination lens arrangement aligned in the second axis into the imaging optical lens arrangement aligned in the first axis to illuminate at least one portion of the retina.

11. The fundoscope of claim 10, wherein the one or more masks and the one or more infrared masks are movable.

12. The fundoscope of claim 1, further comprising:
an illumination source that emits light; and
at least one mask configured to obscure at least some of the light of the illumination source to minimize illumination/reflection intersection within scattering elements of an eye.

13. (canceled)

14. The fundoscope of claim 1, further comprising:
a light source configured to emit infrared light for positioning and/or focus determinations.

15-17. (canceled)

18. The fundoscope of claim 1, wherein the image sensor positioned with the optical lens arrangement and configured to convert detected light to retinal image data comprises:
an image sensor positioned with the optical lens arrangement and configured to convert detected light to retinal image data by capturing multiple high resolution images of adjacent, overlapping, and/or at least partially overlapping areas of a retina.

19-32. (canceled)

33. The fundoscope of claim 1, wherein the at least one communication interface includes a bandwidth capability of approximately one tenth of a capture rate of the retinal image data.

34. (canceled)

35. The fundoscope of claim 1, wherein the obtain the retinal image data from the image sensor comprises:
obtain from the image sensor the retinal image data as a plurality of sequentially captured images of different, adjacent, and/or at least partly overlapping parts of a retina; and
stitch the plurality of sequentially captured images of parts of the retina together to create an overall view.

36. The fundoscope of claim 1, wherein the obtain the retinal image data from the image sensor comprises:
obtain the retinal image data as a plurality of at least partly overlapping images from the image sensor; and
combine the plurality of images into high resolution retinal image data.

37. The fundoscope of claim 1, wherein the generate output data based on analysis of the retinal image data, the output data requiring less bandwidth for transmission than the retinal image data comprises:
generate output data based on analysis of the retinal image data, the output data requiring approximately one tenth in bandwidth for transmission than the retinal image data.

38. (canceled)

39. The fundoscope of claim 1, wherein the generate output data based on analysis of the retinal image data, the output data requiring less bandwidth for transmission than the retinal image data comprises:
generate output data including a reduced resolution version of the retinal image data for transmission.

40. (canceled)

41. The fundoscope of claim 1, wherein the generate output data based on analysis of the retinal image data, the output data requiring less bandwidth for transmission than the retinal image data comprises:
generate output data including a portion of the retinal image data corresponding to a health issue detected based on analysis of the retinal image data.

42. The fundoscope of claim 1, wherein the generate output data based on analysis of the retinal image data, the output data requiring less bandwidth for transmission than the retinal image data comprises:
generate output data including a portion of the retinal image data corresponding to an object or feature detected based on analysis of the retinal image data.

43. The fundoscope of claim 1, wherein the generate output data based on analysis of the retinal image data, the output data requiring less bandwidth for transmission than the retinal image data comprises:
generate output data based on object or feature recognition in the retinal image data, the output data requiring less bandwidth for transmission than the retinal image data.

44-45. (canceled)

46. The fundoscope of claim 1, wherein the generate output data based on analysis of the retinal image data, the output data requiring less bandwidth for transmission than the retinal image data comprises:
generate output data including metadata based on analysis of the retinal image data, the output data requiring less bandwidth for transmission than the retinal image data.

47. (canceled)

48. The fundoscope of claim 1, wherein the generate output data based on analysis of the retinal image data, the output data requiring less bandwidth for transmission than the retinal image data comprises:
generate alphanumeric text output data based on analysis of the retinal image data, the alphanumeric text output data requiring less bandwidth for transmission than the retinal image data.

49. The fundoscope of claim 1, wherein the generate output data based on analysis of the retinal image data, the output data requiring less bandwidth for transmission than the retinal image data comprises:
generate binary output data based on analysis of the retinal image data, the binary output data requiring less bandwidth for transmission than the retinal image data.

50. The fundoscope of claim 1, wherein the generate output data based on analysis of the retinal image data, the output data requiring less bandwidth for transmission than the retinal image data comprises:
generate output data through pixel decimation to maintain a constant resolution independent of a selected area and/or zoom level of the retinal image data.

51-52. (canceled)

53. The fundoscope of claim 1, wherein the generate output data based on analysis of the retinal image data, the output data requiring less bandwidth for transmission than the retinal image data comprises:
generate output data of a specified field of view within the retinal image data, the output data requiring less bandwidth for transmission than the retinal image data.

54. The fundoscope of claim 1, wherein the generate output data based on analysis of the retinal image data, the output data requiring less bandwidth for transmission than the retinal image data comprises:
generate output data of a specified zoom-level within the retinal image data, the output data requiring less bandwidth for transmission than the retinal image data.

55-57. (canceled)

58. The fundoscope of claim 1, wherein the image processor is further programmed to execute an operation including at least:
perform analysis of the retinal image data.

59. The fundoscope of claim 58, wherein the perform analysis of the retinal image data comprises:
obtain baseline retinal image data from the computer readable memory; and
compare the retinal image data to the baseline retinal image data; and
identify at least one deviation between the retinal image data and the baseline retinal image data indicative of at least one health issue.

60. The fundoscope of claim 58, wherein the perform analysis of the retinal image data comprises:
perform object or feature recognition analysis using the retinal image data to identify at least one health issue.

61-64. (canceled)

65. The fundoscope of claim 1, wherein the transmit the output data via the at least one communication interface comprises:
transmit the output data as image data via the at least one communication interface without one or more of static pixels, previously transmitted pixels, or overlapping pixels, wherein the image data is gap filled at a remote server.

66-71. (canceled)

72. The fundoscope of claim 1, wherein the transmit the output data via the at least one communication interface comprises:
transmit the output data via the at least one communication interface in response to detection of at least one health issue and otherwise not transmitting any data.

73. The fundoscope of claim 1, wherein the transmit the output data via the at least one communication interface comprises:
transmit the output data via the at least one communication interface in response to detection of at least one object or feature and otherwise not transmitting any data.

74. The fundoscope of claim 1, wherein the image processor is further programmed to execute an operation comprising:
receive a retinal image analysis application via the at least one communication interface; and
implement the retinal image analysis application with respect to the retinal image data.

75-85. (canceled)

86. A process executed by a computer processor component of a fundoscope that includes an optical lens arrangement, an image sensor configured to convert detected light to retinal image data, and at least one communication interface, the process comprising:
obtain the retinal image data from the image sensor;
generate output data based on analysis of the retinal image data, the output data requiring less bandwidth for transmission than the retinal image data; and
transmit the output data via the at least one communication interface.

87-166. (canceled)

167. A fundoscope comprising:
means for obtaining retinal image data from an image sensor;
means for generating output data based on analysis of the retinal image data, the output data requiring less bandwidth for transmission than the retinal image data; and
means for transmitting the output data via the at least one communication interface.
